(19) United States
(12) Patent Application Publication
(10) Pub. No.: US 2007/0081995 A1
(43) Pub. Date: Apr. 12, 2007

(54) Methods and compositions for targeting IFNAR2

(75) Inventors: Anan Chuntharapai, Colma, CA (US); Kerstin Schmidt, San Francisco, CA (US)

Correspondence Address: GENENTECH, INC., 1 DNA WAY, SOUTH SAN FRANCISCO, CA, 94080, US

(73) Assignee: GENENTECH, INC.

(21) Appl. No.: 11/472,534

(22) Filed: Jun. 21, 2006

Related U.S. Application Data
(60) Provisional application No. 60/692,786, filed on Jun. 22, 2005.

(51) Int. Cl. G01N 33/53; C07H 21/04; C12P 21/06; C12P 21/04; A61K 39/395; C07K 16/24; C12N 5/06

(52) U.S. Cl. 424/145.1; 435/7.1; 435/326; 435/70.21; 530/388.23; 435/69.1; 435/320.1; 536/23.53

(57) ABSTRACT

Anti-IFNAR2 monoclonal antibodies, and methods for using the antibodies, are provided.

### RELATED APPLICATIONS

This application is a non-provisional application filed under 37 CFR 1.53(b)(1), claiming priority under 35 USC 119(e) to provisional application No. 60/692,786 filed Jun. 22, 2005, the contents of which are incorporated herein by reference.

### FIELD OF THE INVENTION

This invention relates to the field of anti-type I interferon receptor antibodies, and more particularly to anti-type I interferon receptor antibodies that block the binding of type I interferons to the second component (IFNAR2) of the type I interferon receptor complex.

### BACKGROUND

The type I interferons (IFNs) are cytokines which have pleiotropic effects on a wide variety of cell types. IFNs are best known for their anti-viral activity, but they also have anti-bacterial, anti-protozoal, immunomodulatory, and cell-growth regulatory functions. The Type I interferons include interferon-α (IFN-α) and interferon-β (IFN-β). Human IFN-α (hIFN-α) is a heterogeneous family with at least 23 polypeptides while there is only one IFN-β polypeptide (J. Interferon Res., 13: 443-444 (1993)). The hIFN-α subtypes show more than 70% amino acid sequence homology, and there is approximately 25% amino acid identity with hIFN-β. The hIFNs-α and hIFN-β share a common receptor.

Two components of the hIFN-α receptor complex have been identified. The cDNA for the first hIFN-α receptor (hIFNAR1) encodes a 63 kD receptor protein (reported in Uze et al., Cell, 60: 225-234 (1990)). This receptor undergoes extensive glycosylation which causes it to migrate in gel electrophoresis as a much larger 135 kD protein. The second interferon receptor, hIFNAR2 (hIFN-αβR long), is a 115 kD protein which mediates a functional signaling complex when associated with hIFNAR1 (reported in Domanski et al., J. Biol. Chem., 270: 21606-21611 (1995)). A variant of IFNAR2, the IFN-α/β receptor (hIFN-αβR short), is a 55 kD protein that can bind to Type I hIFNs but cannot form a functional complex when associated with hIFNAR1 (reported in Novick et al., Cell, 77: 391-400 (1994)). This IFN-α/β receptor appears to be an alternatively spliced variant of hIFNAR2.

The unprocessed hIFNAR1 expression product is composed of 557 amino acids including an extracellular domain (ECD) of 409 residues, a transmembrane domain of 21 residues, and an intracellular domain of 100 residues as shown in FIG. 5 on page 229 of Uze et al., supra. The ECD of IFNAR1 is composed of two domains, domain 1 and domain 2, which are separated by a three-proline motif. There is 19% sequence identity and 50% sequence homology between domains 1 and 2 (Uze et al., supra). Each domain (D200) is composed of approximately 200 residues and can be further subdivided into two homologous subdomains (SD100) of approximately 100 amino acids. The unprocessed hIFNAR2 expression product is composed of 515 amino acids, including an extracellular domain (ECD) of 217 residues, a transmembrane domain of 21 residues, and a long cytoplasmic tail of 250 residues as shown in FIG. 1 on page 21608 of Domanski et al., J. Biol. Chem., 37: 21606-21611 (1995).

Through the use of IFNAR1 gene knockout mice, IFNAR1 has been shown to be essential for the response to all Type I IFNs (Muller et al., Science, 264: 1918-1921 (1994); Cleary et al., J. Biol. Chem., 269: 18747-18749 (1994)) and for the mediation of species-specific IFN signal transduction (Constantinescu et al., Proc. Natl. Acad. Sci. USA, 91: 9602-9606 (1994)). However, IFNAR2, not IFNAR1, plays a crucial role in ligand binding (Cohen et al., Mol. Cell. Biol., 15: 4208 (1995)).

Benoit et al., J. Immunol., 150: 707-716 (1993) reported an anti-IFNAR1 mAb, 64G12, that was found to inhibit the binding of IFN-α2 (IFN-αA) and IFN-αB to Daudi cells and to inhibit the antiviral activity of IFN-α2, IFN-β and IFN-ω(IFN-α_{II}1) on Daudi cells. Benoit et al. also reported that 64G12 recognizes an epitope present in domain 1 of IFNAR1. Eid and Tovey, J. Interferon Cytokine Res., 15: 205-211 (1995) reported that 64G12 cannot immunoprecipitate cross-linked IFN-α2-receptor complexes from Daudi cells.

Colamonici and Domanski, J. Biol. Chem., 268: 10895-10899 (1993) reported an anti-IFNAR2 mAb (denoted the “IFNaRβ1 mAb”) that blocked the binding of IFN-α2 (IFN-αA) to Daudi cells and U-266 cells and blocked the antiproliferative activity of different type I interferons on Daudi cells using MTT cell proliferation assays.

Various other antibodies that interfere with the Type I interferon-interferon receptor interaction have also been disclosed. See, for example, U.S. Pat. Nos. 5,516,515, 5,919,453, 5,643,749, 5,821,078, 5,886,153, 6,458,932, 6,136,309, 6,713,609, 6,787,634, WO9320187, WO96/33735, EP0822830, EP495907, WO 95/07716, WO96/34096, EP 0537166 B1, EP588177 A2, EP588177 B 1, WO9741229, EP927252, EP676413 B1, WO2004/093908, WO2004/094473, and US Pub. 2003/0018174, US Pub. 2003/0166228.

The roles played by the Type I interferon pathway in various diseases are beginning to be understood. These diseases include many manifestations of immune complex dysregulation. See, e.g., Schmidt & Ouyang, Lupus (2004), 13:348-352. It is clear that it would be beneficial to have compositions and methods that are effective in targeting and modulating this important pathway. The invention provided herein relate to such compositions and methods.

All references cited herein, including patent applications and publications, are incorporated by reference in their entirety.

### DISCLOSURE OF THE INVENTION

The invention provides novel antibodies capable of binding IFNAR2 and/or regulating biological activities associated with Type I interferon signaling through the second component (IFNAR2) of the type I interferon receptor complex.

In one aspect, the invention provides an isolated immunoglobulin polypeptide comprising at least one, two, three, four, five or all hypervariable (HVR) sequences selected from the group consisting of HC-HVR1, HC-HVR2, HC-HVR3, LC-HVR1, LC-HVR2 and LC-HVR3 sequence of an antibody produced by hybridoma cell line deposited at American Type Culture Collection (ATCC) under Accession No. PTA-6242, PTA-6243 or PTA-6244, wherein said isolated immunoglobulin polypeptide specifically binds human IFNAR2. For example, in one aspect, the invention provides an isolated antibody comprising at least one, two, three, four, five or all hypervariable (HVR) sequences selected from the group consisting of HC-HVR1, HC-HVR2, HC-HVR3, LC-HVR1, LC-HVR2 and LC-HVR3 sequence of an antibody produced by hybridoma cell line deposited at American Type Culture Collection (ATCC) under Accession No. PTA-6242, PTA-6243 or PTA-6244, wherein said isolated antibody specifically binds human IFNAR2. In one embodiment, the invention provides an isolated antibody comprising at least one, two or all of HC-HVR selected from the group consisting of HC-HVR1, HC-HVR2 and HC-HVR3, and at least one, two or all of LC-HVR selected from the group consisting of LC-HVR1, LC-HVR2 and LC-HVR3. In one embodiment, the HVR sequences in an isolated antibody of the invention are those of an antibody produced by hybridoma cell line deposited at American Type Culture Collection (ATCC) under Accession No. PTA-6242. In one embodiment, the HVR sequences in an isolated antibody of the invention are those of an antibody produced by hybridoma cell line deposited at American Type Culture Collection (ATCC) under Accession PTA-6243. In one embodiment, the HVR sequences in an isolated antibody of the invention are those of an antibody produced by hybridoma cell line deposited at American Type Culture Collection (ATCC) under Accession No. PTA-6244.

In one aspect, the invention provides an isolated immunoglobulin polypeptide comprising heavy and/or light chain variable domain sequence of an antibody produced by hybridoma cell line deposited at American Type Culture Collection (ATCC) under Accession No. PTA-6242, PTA-6243 or PTA-6244, wherein said isolated immunoglobulin polypeptide specifically binds human IFNAR2. For example, in one aspect, the invention provides an isolated antibody comprising heavy and/or light chain variable domain sequence of an antibody produced by hybridoma cell line deposited at American Type Culture Collection (ATCC) under Accession No. PTA-6242, PTA-6243 or PTA-6244, wherein said isolated antibody specifically binds human IFNAR2. In one embodiment, the isolated antibody comprises heavy and/or light chain variable domain sequence of an antibody produced by hybridoma cell line deposited at American Type Culture Collection (ATCC) under Accession No. PTA-6242. In one embodiment, the isolated antibody comprises heavy and/or light chain variable domain sequence of an antibody produced by hybridoma cell line deposited at American Type Culture Collection (ATCC) under Accession No. PTA-6243. In one embodiment, the isolated antibody comprises heavy and/or light chain variable domain sequence of an antibody produced by hybridoma cell line deposited at American Type Culture Collection (ATCC) under Accession No. PTA-6244.

In one aspect, the invention provides an IFNAR2 antibody encoded by an antibody coding sequence of hybridoma cell line deposited at the American Type Culture Collection (ATCC) under Accession No. PTA-6242, PTA-6243 or PTA-6244.

In one aspect, the invention provides an isolated antibody that binds to the same epitope on human IFNAR2 as an antibody produced by hybridoma cell line deposited at American Type Culture Collection (ATCC) under Accession No. PTA-6242, PTA-6243 and/or PTA-6244.

In one aspect, the invention provides an isolated antibody that competes with an antibody produced by hybridoma cell line deposited at American Type Culture Collection (ATCC) under Accession No. PTA-6242, PTA-6243 and/or PTA-6244 for binding to human IFNAR2.

In one embodiment of an antibody of the invention, the antibody inhibits anti-viral activity of human leukocyte interferon.

In one embodiment of an antibody of the invention, the antibody inhibits anti-viral activity of human interferon alpha.

In one embodiment of an antibody of the invention, at least about 10 ug/ml of the antibody in full length IgG form inhibits at least about 25%, 40%, 50%, 75%, or 90% of anti-viral activity of from about 0.5 U/ml to about 1000 U/ml of human leukocyte interferon. In one embodiment, the leukocyte interferon is about 10 U/ml.

In one embodiment of an antibody of the invention, at least about 10 ug/ml of the antibody in full length IgG form inhibits at least about 25%, 40%, 50%, 75%, or 90% of anti-viral activity of about 1000 U/ml of interferon α.

In one embodiment of an antibody of the invention, at least about 0.01, 0.04, 0.1, 0.4, 1.1, 3.3, 10 or 20 ug/ml of the antibody in full length IgG form inhibits at least about 25%, 40%, 50%, 75%, or 90% of anti-viral activity of about 25 U/ml of interferon β. In one embodiment, the antibody concentration is at least about 10 ug/ml. In one embodiment, at least about 10 ug/ml of an antibody of the invention in full length IgG form inhibits at least about 25% of anti-viral activity of about 25 U/ml of interferon β.

In one embodiment of an antibody of the invention, the full length IgG form of the antibody specifically binds human IFNAR2 with a binding affinity of about 300 pM or better. In one embodiment, the binding affinity is about 280 pM or better. In one embodiment, the binding affinity is about 200 pM or better. In one embodiment, the binding affinity is about 100 pM or better. In one embodiment, the binding affinity is about 60 pM or better.

In one embodiment, an antibody of the invention blocks anti-viral activity of interferon α and interferon β at substantially equivalent antibody titer.

In one embodiment, an antibody of the invention has substantially equivalent potency in vitro in blocking anti-viral activity of a first Type I interferon (e.g., interferon α) and a second Type I interferon (e.g., interferon β. For example, in one embodiment, an equivalent amount of an antibody of the invention is capable of blocking at least about 50%, 75%, 85%, 90% or 95% of anti-viral activity of a first Type I interferon and a second Type I interferon, wherein the interferons are each administered at their respective approximate optimal anti-viral amount in a WISH cell bioassay (e.g., as described in Examples below), and wherein the second Type I interferon is interferon β. In one embodiment, the first Type I interferon is interferon α. In one embodiment, the first Type I interferon is leukocyte interferon.

An antibody of the invention can be in any number of forms. For example, an antibody of the invention can be a chimeric antibody, a humanized antibody or a human antibody. In one embodiment, an antibody of the invention is not a human antibody, for example it is not an antibody produced in a xenomouse (e.g., as described in WO96/33735). An antibody of the invention can be full length or a fragment thereof (e.g., a fragment comprising an antigen binding component).

In one embodiment, an antibody of the invention is not an antibody produced by hybridoma cell line having ATCC Deposit No. HB-12426, 12427 and/or 12428, or an IFNAR2 antibody described on pages 10895 to 10899 in Journal of Biological Chemistry, Volume 268 published in 1993, or an isolated IFNAR2 antibody disclosed in PCT Publications WO96/33735, WO96/34096, WO9741229, European Patent Nos. 588177 B1, 927252, 676413, and/or U.S. Pat. Nos. 6,458,932 and 6,136,309.

In one embodiment, an antibody of the invention does not compete for binding to human IFNAR2 with an antibody produced by hybridoma cell line having ATCC Deposit No. HB-12426, 12427 and/or 12428, or an IFNAR2 antibody described on pages 10895 to 10899 in Journal of Biological Chemistry, Volume 268 published in 1993, or an isolated IFNAR2 antibody disclosed in PCT Publications WO96/33735, WO96/34096, WO9741229, European Patent Nos. 588177 B1, 927252, 676413, and/or U.S. Pat. Nos. 6,458,932 and 6,136,309.

In one embodiment, an antibody of the invention does not bind to the same epitope on human IFNAR2 as an antibody produced by hybridoma cell line having ATCC Deposit No. HB-12426, 12427 and/or 12428, or an IFNAR2 antibody described on pages 10895 to 10899 in Journal of Biological Chemistry, Volume 268 published in 1993, or an isolated IFNAR2 antibody disclosed in PCT Publications WO96/33735, WO96/34096, WO9741229, European Patent Nos. 588177 B1, 927252, 676413, and/or U.S. Pat. Nos. 6,458,932 and 6,136,309.

In one aspect, the invention provides compositions comprising one or more antibodies of the invention and a carrier. In one embodiment, the carrier is pharmaceutically acceptable.

In one aspect, the invention provides nucleic acids encoding an immunoglobulin polypeptide (e.g., an antibody) of the invention.

In one aspect, the invention provides vectors comprising a nucleic acid of the invention.

In one aspect, the invention provides host cells comprising a nucleic acid or a vector of the invention. A vector can be of any type, for example a recombinant vector such as an expression vector. Any of a variety of host cells can be used. In one embodiment, a host cell is a prokaryotic cell, for example, E. coli. In one embodiment, a host cell is a eukaryotic cell, for example a mammalian cell such as Chinese Hamster Ovary (CHO) cell.

In one aspect, the invention provides methods for making an antibody of the invention. For example, the invention provides a method of making an anti-IFNAR2 antibody (which, as defined herein includes full length and fragments thereof), said method comprising expressing in a suitable host cell a recombinant vector of the invention encoding said antibody (or fragment thereof), and recovering said antibody.

In one aspect, the invention provides an article of manufacture comprising a container; and a composition contained within the container, wherein the composition comprises one or more antibodies of the invention. In one embodiment, the composition comprises a nucleic acid of the invention. In one embodiment, a composition comprising an immunoglobulin polypeptide (e.g. an antibody) of the invention further comprises a carrier, which in some embodiments is pharmaceutically acceptable. In one embodiment, an article of manufacture of the invention further comprises instructions for administering the composition (e.g., the antibody) to a subject.

In one aspect, the invention provides a kit comprising a first container comprising a composition comprising one or more antibodies of the invention; and a second container comprising a buffer. In one embodiment, the buffer is pharmaceutically acceptable. In one embodiment, a composition comprising an antibody further comprises a carrier, which in some embodiments is pharmaceutically acceptable. In one embodiment, a kit further comprises instructions for administering the composition (e.g., the antibody) to a subject.

In one aspect, the invention provides use of an antibody of the invention in the preparation of a medicament for the therapeutic and/or prophylactic treatment of a disease, such as a cell proliferative disorder or an immune (such as autoimmune) disorder.

In one aspect, the invention provides use of a nucleic acid of the invention in the preparation of a medicament for the therapeutic and/or prophylactic treatment of a disease, such as a cell proliferative disorder or an immune (such as autoimmune) disorder.

In one aspect, the invention provides use of an expression vector of the invention in the preparation of a medicament for the therapeutic and/or prophylactic treatment of a disease, such as a cell proliferative disorder or an immune (such as autoimmune) disorder.

In one aspect, the invention provides use of a host cell of the invention in the preparation of a medicament for the therapeutic and/or prophylactic treatment of a disease, such as a cell proliferative disorder or an immune (such as autoimmune) disorder.

In one aspect, the invention provides use of an article of manufacture of the invention in the preparation of a medicament for the therapeutic and/or prophylactic treatment of a disease, such as a cell proliferative disorder or an immune (such as autoimmune) disorder.

In one aspect, the invention provides use of a kit of the invention in the preparation of a medicament for the therapeutic and/or prophylactic treatment of a disease, such as a cell proliferative disorder or an immune (such as autoimmune) disorder.

The invention provides methods and compositions useful for modulating disease states associated with dysregulation of the Type I interferon/IFNAR2 signaling axis. This signaling pathway is involved in multiple biological and physiological functions. Antibodies of the invention are capable of modulating this pathway, and therefore are useful for modulating conditions associated with aberrations in one or more of these biological and physiological functions. Thus, in one aspect, the invention provides a method comprising administering to a subject an antibody of the invention, whereby a pathological condition is treated.

In one aspect, the invention provides a method for treatment of a disease or condition associated with overexpression and/or abnormally high level of activity of IFN-α, β and/or IFNAR2, the method comprising administering to a subject an effective amount of an antibody of the invention, whereby the disease/condition is treated. In one embodiment, the subject is a mammal. In one embodiment, the subject is human.

Methods and compositions of the invention can be used to treat a variety of diseases associated with overexpression and/or abnormally high level of activity of IFN-α, β and/or IFNAR2. For example, in one embodiment, a disease treated by a method or composition of the invention is an autoimmune disease, for example insulin-dependent diabetes mellitus (IDDM); systemic lupus erythematosus (SLE) (which may include, e.g., lupus nephritis), autoimmune thyroiditis, Sjogren's syndrome, psoriasis, inflammatory bowel disease (e.g., ulcerative colitis, Crohn's disease), rheumatoid arthritis and IgA nephropathy.

In one aspect, the invention provides a method of inhibiting Type I interferon/IFNAR2 signaling in a cell or tissue, said method comprising contacting the cell or tissue with an effective amount of an antibody of the invention, whereby Type I interferon/IFNAR2 signaling in the cell or tissue is inhibited.

In one aspect, the invention provides a method of treating a pathological condition associated with dysregulation of Type I interferon/IFNAR2 cell signaling in a subject, said method comprising administering to the subject an effective amount of an antibody of the invention, whereby said condition is treated. In one embodiment, the pathological condition is associated with upregulation of Type I interferon/IFNAR2 expression.

Methods of the invention can be used to affect any suitable pathological state, for example, cells and/or tissues associated with dysregulation of the Type I interferon/IFNAR2 signaling pathway. In one embodiment, a cell that is targeted in a method of the invention is an immune cell. In one embodiment, the immune cell is a T-cell, B-cell or monocyte.

In one embodiment, inhibition of Type I interferon/IFNAR2 cell signaling by an antibody of the invention is associated with inhibition of signaling through Tyk2, Jak1, Stat1 and/or Stat2. In one embodiment, inhibition of Type I interferon/IFNAR2 cell signaling by an antibody of the invention is associated with inhibition of formation of ISRE complex. In one embodiment, inhibition of Type I interferon/IFNAR2 cell signaling by an antibody of the invention is associated with inhibition of expression of interferon-regulated genes (e.g., Mx-1, MHC I, CD69, Fas).

Methods of the invention can further comprise additional treatment steps/agents. For example, in one embodiment, a patient may also be administered a steroid (e.g., for an autoimmune disease).

In one aspect, an antibody of the invention is linked to a toxin such as a cytotoxic agent. These molecules can be formulated or administered in combination with an additive/enhancing agent, such as a steroid.

In one aspect, the invention provides a method of detecting presence of IFNAR2 in a sample, comprising contacting the sample with an antibody of the invention.

In one aspect, the invention provides a method of diagnosing a disease comprising determining the level of IFNAR2 in a test sample of tissue cells by contacting the sample with an antibody of the invention, whereby IFNAR2 bound by the antibody indicates presence and/or amount of IFNAR2 in the sample. In one aspect, the invention provides a method of diagnosing a disease comprising determining the level of Type I interferon/IFNAR2 biological activity in a test sample of tissue cells by contacting the sample with an antibody of the invention, whereby a decrease of said biological activity in the sample compared to a control sample indicates presence and/or an increased level of Type I interferon/IFNAR2 biological activity in the test sample.

In another aspect, the invention provides a method of determining whether an individual is at risk for a disease comprising determining the level of IFNAR2 in a test sample of tissue cell by contacting the test sample with an antibody of the invention and thereby determining the amount of IFNAR2 present in the sample, wherein a higher level of IFNAR2 in the test sample, as compared to a control sample comprising normal tissue of the same cell origin as the test sample, is an indication that the individual is at risk for the disease.

In one embodiment of methods of the invention, the level of IFNAR2 is determined based on amount of IFNAR2 polypeptide indicated by amount of IFNAR2 bound by the antibody in the test sample. An antibody employed in the method may optionally be detectably labeled, attached to a solid support, or the like. In one embodiment of methods of the invention, the amount of inhibition of Type I interferon/IFNAR2 biological activity is determined based on amount of biological activity associated with signaling through the Type I interferon/IFNAR2 pathway, for example through inhibition of signaling through Tyk2, Jak1, Stat1 and/or Stat2; through inhibition of ISRE complex formation, and/or through inhibition of expression of IFN-regulated genes.

In one aspect, the invention provides a method of binding an antibody of the invention to IFNAR2 present in a bodily fluid, for example blood.

In yet another aspect, the invention is directed to a method of binding an antibody of the invention to a cell that expresses IFNAR2, wherein the method comprises contacting said cell with said antibody under conditions which are suitable for binding of the antibody to IFNAR2 and allowing binding therebetween. In one embodiment, binding of said antibody to IFNAR2 on the cell inhibits an IFNAR2 biological function. In one embodiment, said antibody does not inhibit interaction of IFNAR2 with its ligand. In one embodiment, said antibody binds to an IFNAR2 molecule on the cell and inhibits binding of another molecule to the IFNAR2 molecule.

In one aspect, the invention provides a method of targeting a therapeutic agent to an IFNAR2-associated tissue in a host, the method comprising administering to the host said therapeutic agent in a form that is linked to an antibody of the invention, whereby the agent is targeted to the IFNAR2-associated tissue in the host. In one embodiment, the antibody that binds IFNAR2 is capable of specifically binding to IFNAR2 located on a cell (either in vitro or in vivo), for example where IFNAR2 is present on the surface of a cell.

### BRIEF DESCRIPTION OF THE DRAWINGS

FIG. 1 shows a graphical depiction of data from a WISH interferon bioassay in which neutralization effect of antibodies 1922 and 1923 was assessed over a range of interferon α concentrations.

FIG. 2 shows a graphical depiction of data from a WISH interferon bioassay in which neutralization effect of antibody 1922 was assessed. Effect was assessed over either a range of human leukocyte interferon concentrations or a range of antibody concentrations.

FIG. 3 shows a graphical depiction of data from a WISH interferon bioassay in which neutralization effect of antibody 1923 was assessed. Effect was assessed over either a range of human leukocyte interferon concentrations or a range of antibody concentrations.

FIG. 4 shows a graphical depiction of data from a WISH interferon bioassay in which neutralization effect of antibodies 1922 and 1923 was assessed against interferon α or interferon β.

FIG. 5 shows a graphical depiction of data from a WISH interferon bioassay in which neutralization effect of antibody 1922 was assessed over a range of interferon β concentrations.

FIG. 6 shows a graphical depiction of data from a WISH interferon bioassay in which neutralization effect of antibody 1922 was assessed over a range of antibody concentrations.

FIG. 7 shows a graphical depiction of data from a WISH interferon bioassay in which neutralization effect of antibody 1923 was assessed over a range of antibody concentrations.

### MODES FOR CARRYING OUT THE INVENTION

General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

Definitions

As used herein, the terms “type I interferon” and “human type I interferon” are defined as all species of native human and synthetic interferon which fall within the human and synthetic interferon-α, interferon-ω and interferon-β classes and which bind to a common cellular receptor. Natural human interferon-α comprises 23 or more closely related proteins encoded by distinct genes with a high degree of structural homology (Weissmann and Weber, Prog. Nucl. Acid. Res. Mol. Biol., 33: 251 (1986); J. Interferon Res., 13: 443-444 (1993)). The human IFN-α locus comprises two subfamilies. The first subfamily consists of at least 14 functional, non-allelic genes, including genes encoding IFN-αA (IFN-α2), IFN-αB (IFN-α8), IFN-α (IFN-α10), IFN-αD (IFN-α1), IFN-αE (IFN-α22), IFN-αF (IFN-α21), IFN-αG (IFN-α5), IFN-α16, IFN-α17, IFN-α4, IFN-α6, IFN-α7, and IFN-αH (IFN-α14), and pseudogenes having at least 80% homology. The second subfamily, α_{II}1 or ω, contains at least 5 pseudogenes and 1 functional gene (denoted herein as “IFN-α_{II}1” or “IFN-ω”) which exhibits 70% homology with the IFN-α genes (Weissmann and Weber (1986)). The human IFN-β is generally thought to be encoded by a single copy gene.

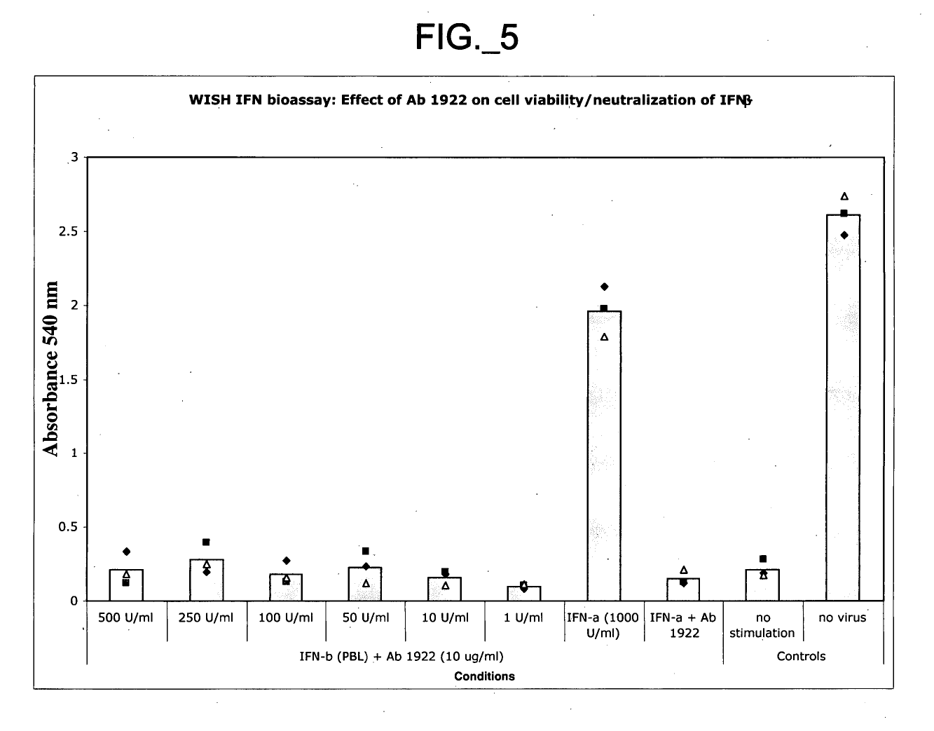

As used herein, the terms “first human interferon-α (hIFN-α) receptor”, “IFN-αR”, “hIFNAR1”, “IFNAR1”, and “Uze chain” are defined as the 557 amino acid receptor protein cloned by Uze et al., Cell, 60: 225-234 (1990), including an extracellular domain of 409 residues, a transmembrane domain of 21 residues, and an intracellular domain of 100 residues, as shown in FIG. 5 on page 229 of Uze et al. In one embodiment, the foregoing terms include fragments of IFNAR1 that contain the extracellular domain (ECD) (or fragments of the ECD) of IFNAR1.

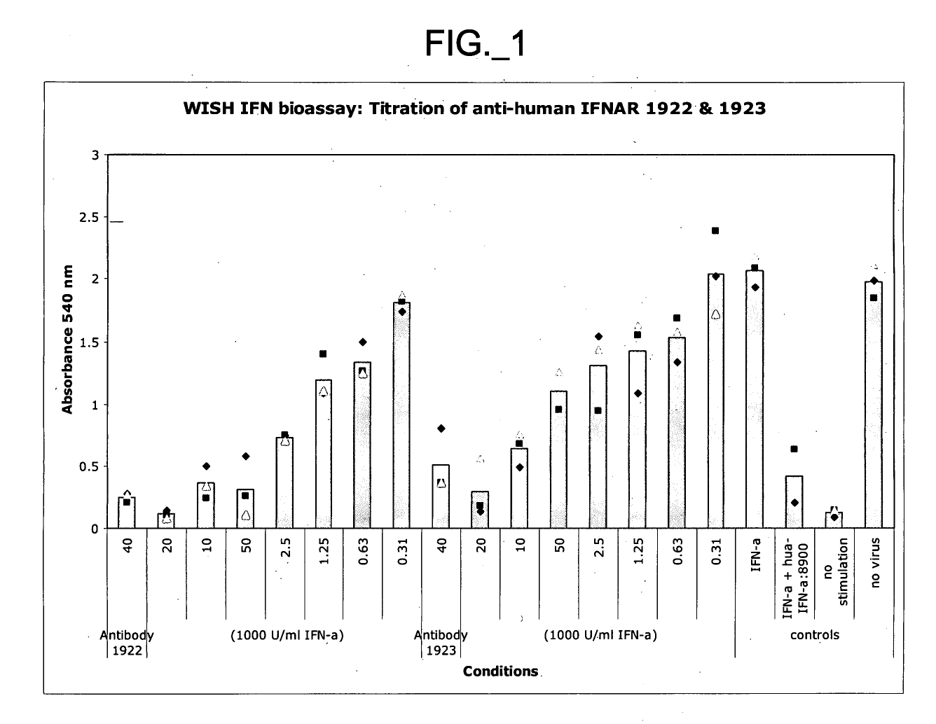

As used herein, the terms “second human interferon-α (hIFN-α) receptor”, “IFN-αβR”, “hIFNAR2”, “IFNAR2”, and “Novick chain” are defined as the 515 amino acid receptor protein cloned by Domanski et al., J. Biol. Chem., 37: 21606-21611 (1995), including an extracellular domain of 217 residues, a transmembrane domain of 21 residues, and an intracellular domain of 250 residues, as shown in FIG. 1 on page 21608 of Domanski et al. In one embodiment, the foregoing terms include fragments of IFNAR2 that contain the extracellular domain (ECD) (or fragments of the ECD) of IFNAR2, and soluble forms of IFNAR2, such as IFNAR2 ECD fused to at least a portion of an immunoglobulin sequence.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In one embodiment, the antibody will be purified (1) to greater than 95% by weight of antibody as determined by, for example, the Lowry method, and in some embodiments more than 99% by weight, (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

As used herein, the term “anti-IFNAR2 antibody” refers to an antibody that is capable of binding to IFNAR2.

As used herein, an antibody of the invention with the property or capability of “blocking the binding of a type I interferon to IFNAR2” is defined as an anti-IFNAR2 antibody capable of binding to IFNAR2 such that the ability of IFNAR2 to bind to one or more type I interferons is impaired or eliminated.

The phrase “substantially similar,” “substantially the same”, “equivalent”, or “substantially equivalent”, as used herein, denotes a sufficiently high degree of similarity between two numeric values (for example, one associated with a molecule and the other associated with a reference/comparator molecule) such that one of skill in the art would consider the difference between the two values to be of little or no biological and/or statistical significance within the context of the biological characteristic measured by said values (e.g., Kd values, anti-viral effects, etc.). The difference between said two values is preferably less than about 50%, preferably less than about 40%, preferably less than about 30%, preferably less than about 20%, preferably less than about 10% as a function of the value for the reference/comparator molecule.

The phrase “substantially reduced,” or “substantially different”, as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values, HAMA response, anti-viral activity). The difference between said two values is preferably greater than about 10%, preferably greater than about 20%, preferably greater than about 30%, preferably greater than about 40%, preferably greater than about 50% as a function of the value for the reference/comparator molecule.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described in the following.

In one embodiment, the “Kd” or “Kd value” according to this invention is measured by a radiolabeled antigen binding assay (RIA) performed with the Fab version of an antibody of interest and its antigen as described by the following assay that measures solution binding affinity of Fabs for antigen by equilibrating Fab with a minimal concentration of (¹²⁵I)-labeled antigen in the presence of a titration series of unlabeled antigen, then capturing bound antigen with an anti-Fab antibody-coated plate (Chen, et al., (1999) J. Mol Biol 293:865-881). To establish conditions for the assay, microtiter plates (Dynex) are coated overnight with 5 ug/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbant plate (Nunc #269620), 100 pM or 26 pM [¹²⁵I]-antigen are mixed with serial dilutions of a Fab of interest (e.g., consistent with assessment of an anti-VEGF antibody, Fab-12, in Presta et al., (1997) Cancer Res. 57:4593-4599). The Fab of interest is then incubated overnight; however, the incubation may continue for a longer period (e.g., 65 hours) to insure that equilibrium is reached. Thereafter, the mixtures are transferred to the capture plate for incubation at room temperature (e.g., for one hour). The solution is then removed and the plate washed eight times with 0.1% Tween-20 in PBS. When the plates have dried, 150 ul/well of scintillant (MicroScint-20; Packard) is added, and the plates are counted on a Topcount gamma counter (Packard) for ten minutes. Concentrations of each Fab that give less than or equal to 20% of maximal binding are chosen for use in competitive binding assays. According to another embodiment the Kd or Kd value is measured by using surface plasmon resonance assays using a BIAcore™-2000 or a BIAcore™-3000 (BIAcore, Inc., Piscataway, N.J.) at 25C with immobilized antigen CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, into 5 ug/ml (˜0.2 uM) before injection at a flow rate of 5 ul/minute to achieve approximately 10 response units (RU) of coupled protein. Following the injection of antigen, 1M ethanolamine is injected to block unreacted groups. For kinetics measurements, two-fold serial dilutions of Fab (0.78 nM to 500 nM) are injected in PBS with 0.05% Tween 20 (PBST) at 25° C. at a flow rate of approximately 25 μl/min. Association rates (kₒₙ) and dissociation rates (k_{off}) are calculated using a simple one-to-one Langmuir binding model (BIAcore Evaluation Software version 3.2) by simultaneous fitting the association and dissociation sensorgram. The equilibrium dissociation constant (Kd) is calculated as the ratio k_{off}/kₒₙ. See, e.g., Chen, Y., et al., (1999) J. Mol Biol 293:865-881. If the on-rate exceeds 10⁶ M⁻¹ S⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-Aminco spectrophotometer (ThermoSpectronic) with a stir red cuvette.

An “on-rate” or “rate of association” or “association rate” or “kₒₙ” according to this invention can also be determined with the same surface plasmon resonance technique described above using a BIAcore™-2000 or a BIAcore™-3000 (BIAcore, Inc., Piscataway, N.J.) at 25C with immobilized antigen CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, into 5 ug/ml (˜0.2 uM) before injection at a flow rate of 5 ul/minute to achieve approximately 10 response units (RU) of coupled protein. Following the injection of 1M ethanolamine to block unreacted groups. For kinetics measurements, two-fold serial dilutions of Fab (0.78 nM to 500 nM) are injected in PBS with 0.05% Tween 20 (PBST) at 25° C. at a flow rate of approximately 25 ul/min. Association rates (kₒₙ) and dissociation rates (k_{off}) are calculated using a simple one-to-one Langmuir binding model (BIAcore Evaluation Software version 3.2) by simultaneous fitting the association and dissociation sensorgram. The equilibrium dissociation constant (Kd) was calculated as the ratio k_{off}/kₒₙ. See, e.g., Chen, Y., et al., (1999) J. Mol Biol 293:865-881. However, if the on-rate exceeds 10⁶ M⁻¹ S⁻¹ by the surface plasmon resonance assay above, then the on-rate is preferably determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a a spectrometer, such as a stop-flow equipped spectrophometer (Aviv Instruments) or a 8000-series SLM-Aminco spectrophotometer (ThermoSpectronic) with a stirred cuvette. The “Kd” or “Kd value” according to this invention is in one embodiment measured by a radiolabeled antigen binding assay (RIA) performed with the Fab version of the antibody and antigen molecule as described by the following assay that measures solution binding affinity of Fabs for antigen by equilibrating Fab with a minimal concentration of (¹²⁵I)-labeled antigen in the presence of a titration series of unlabeled antigen, then capturing bound antigen with an anti-Fab antibody-coated plate (Chen, et al., (1999) J. Mol Biol 293:865-881). To establish conditions for the assay, microtiter plates (Dynex) are coated overnight with 5 ug/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbant plate (Nunc #269620), 100 pM or 26 pM [¹²⁵I]-antigen are mixed with serial dilutions of a Fab of interest (consistent with assessement of an anti-VEGF antibody, Fab-12, in Presta et al., (1997) Cancer Res. 57:4593-4599). The Fab of interest is then incubated overnight; however, the incubation may continue for a longer period (e.g., 65 hours) to insure that equilibrium is reached. Thereafter, the mixtures are transferred to the capture plate for incubation at room temperature for one hour. The solution is then removed and the plate washed eight times with 0.1% Tween-20 in PBS. When the plates have dried, 150 ul/well of scintillant (MicroScint-20; Packard) is added, and the plates are counted on a Topcount gamma counter (Packard) for ten minutes. Concentrations of each Fab that give less than or equal to 20% of maximal binding are chosen for use in competitive binding assays. According to another embodiment, the Kd or Kd value is measured by using surface plasmon resonance assays using a BIAcore™-2000 or a BIAcore™-3000 (BIAcore, Inc., Piscataway, N.J.) at 25C with immobilized antigen CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, into 5 ug/ml (˜0.2 uM) before injection at a flow rate of 5 ul/minute to achieve approximately 10 response units (RU) of coupled protein. Following the injection of antigen, 1M ethanolamine is injected to block unreacted groups. For kinetics measurements, two-fold serial dilutions of Fab (0.78 nM to 500 nM) are injected in PBS with 0.05% Tween 20 (PBST) at 25° C. at a flow rate of approximately 25 ul/min. Association rates (kₒₙ) and dissociation rates (k_{off}) are calculated using a simple one-to-one Langmuir binding model (BIAcore Evaluation Software version 3.2) by simultaneous fitting the association and dissociation sensorgram. The equilibrium dissociation constant (Kd) is calculated as the ratio k_{off}/kₒₙ. See, e.g., Chen, Y., et al., (1999) J. Mol Biol 293:865-881. If the on-rate exceeds 10⁶ M⁻¹ S⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-Aminco spectrophotometer (ThermoSpectronic) with a stir red cuvette.

In one embodiment, an “on-rate” or “rate of association” or “association rate” or “kₒₙ ” according to this invention is determined with the same surface plasmon resonance technique described above using a BIAcore™-2000 or a BIAcore™-3000 (BIAcore, Inc., Piscataway, N.J.) at 25C with immobilized antigen CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, into 5 ug/ml (˜0.2 uM) before injection at a flow rate of 5 ul/minute to achieve approximately 10 response units (RU) of coupled protein. Following the injection of 1M ethanolamine to block unreacted groups. For kinetics measurements, two-fold serial dilutions of Fab (0.78 nM to 500 nM) are injected in PBS with 0.05% Tween 20 (PBST) at 25° C. at a flow rate of approximately 25 ul/min. Association rates (kₒₙ) and dissociation rates (k_{off}) are calculated using a simple one-to-one Langmuir binding model (BIAcore Evaluation Software version 3.2) by simultaneous fitting the association and dissociation sensorgram. The equilibrium dissociation constant (Kd) was calculated as the ratio k_{off}/kₒₙ. See, e.g., Chen, Y., et al., (1999) J. Mol Biol 293:865-881. However, if the on-rate exceeds 10⁶ M⁻¹ S⁻¹ by the surface plasmon resonance assay above, then the on-rate is preferably determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a a spectrometer, such as a stop-flow equipped spectrophometer (Aviv Instruments) or a 8000-series SLM-Aminco spectrophotometer (ThermoSpectronic) with a stirred cuvette.

The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors”). In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which generally lack antigen specificity. Polypeptides of the latter kind are, for example, produced at low levels by the lymph system and at increased levels by myelomas.

The terms “antibody” and “immunoglobulin” are used interchangeably in the broadest sense and include monoclonal antibodies (e.g., full length or intact monoclonal antibodies), polyclonal antibodies, monovalent, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

Depending on the amino acid sequences of the constant domains of their heavy chains, antibodies (immunoglobulins) can be assigned to different classes. There are five major classes of immunoglobulins: IgA, IgD, IgE, IgG and IgM, and several of these may be further divided into subclasses (isotypes), e.g., IgG-1, IgG-2, IgA-1, IgA-2, and etc. The heavy chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al. Cellular and Mol. Immunology, 4th ed. (2000). An antibody may be part of a larger fusion molecule, formed by covalent or non-covalent association of the antibody with one or more other proteins or peptides.

The terms “full length antibody,” “intact antibody” and “whole antibody” are used herein interchangeably, to refer to an antibody in its substantially intact form, not antibody fragments as defined below. The terms particularly refer to an antibody with heavy chains that contain the Fc region.

“Antibody fragments” comprise only a portion of an intact antibody, wherein the portion preferably retains at least one, preferably most or all, of the functions normally associated with that portion when present in an intact antibody. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half life modulation, ADCC function and complement binding. In one embodiment, an antibody fragment is a monovalent antibody that has an in vivo half life substantially similar to an intact antibody. For example, such an antibody fragment may comprise on antigen binding arm linked to an Fc sequence capable of conferring in vivo stability to the fragment.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region of the recipient are replaced by residues from a hypervariable region of a non-human species (donor antibody) such as mouse, rat, rabbit or nonhuman primate having the desired specificity, affinity, and/or capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma& Immunol. 1: 105-115 (1998); Harris, Biochem. Soc. Transactions 23:1035-1038 (1995); Hurle and Gross, Curr. Op. Biotech. 5:428-433 (1994).

The term “hypervariable region”, “HVR”, or “HV”, when used herein refers to the regions of an antibody variable domain which are hypervariable in sequence and/or form structurally defined loops. The letters “HC” and “LC” preceding the term “HVR” or “HV” refers, respectively, to HVR or HV of a heavy chain and light chain. Generally, antibodies comprise six hypervariable regions; three in the VH (H1, H2, H3), and three in the VL (L1, L2, L3). A number of hypervariable region delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM hypervariable regions represent a compromise between the Kabat CDRs and Chothia structural loops, and are used by Oxford Molecular's AbM antibody modeling software. The “contact” hypervariable regions are based on an analysis of the available complex crystal structures. The residues from each of these hypervariable regions are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |

(Kabat Numbering)

| | | | | |
|---|---|---|---|---|
| H1 | H31-H35 | H26-H35 | H26-H32 | H30-H35 |

(Chothia Numbering)

| | | | | |
|---|---|---|---|---|
| H2 | H50-H65 | H50-H58 | H53-H55 | H47-H58 |
| H3 | H95-H102 | H95-H102 | H96-H101 | H93-H101 |

“Framework” or “FR” residues are those variable domain residues other than the hypervariable region residues as herein defined.

The “variable region” or “variable domain” of an antibody refers to the amino-terminal domains of heavy or light chain of the antibody. These domains are generally the most variable parts of an antibody and contain the antigen-binding sites.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues.

An “affinity matured” antibody is one with one or more alterations in one or more HVRs thereof which result in an improvement in the affinity of the antibody for antigen, compared to a parent antibody which does not possess those alteration(s). In one embodiment, an affinity matured antibody has nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

A “blocking” antibody or an “antagonist” antibody is one which inhibits or reduces biological activity of the antigen it binds. Preferred blocking antibodies or antagonist antibodies substantially or completely inhibit the biological activity of the antigen.

An “agonist antibody”, as used herein, is an antibody which mimics at least one of the functional activities of a polypeptide of interest.

A “disorder” is any condition that would benefit from treatment with an antibody of the invention. This includes chronic and acute disorders or diseases including those pathological conditions which predispose the mammal to the disorder in question. Non-limiting examples of disorders to be treated herein include inflammatory, immunologic and other interferon-related disorders.

An “autoimmune disease” herein is a non-malignant disease or disorder arising from and directed against an individual's own tissues. The autoimmune diseases herein specifically exclude malignant or cancerous diseases or conditions, especially excluding B cell lymphoma, acute lymphoblastic leukemia (ALL), chronic lymphocytic leukemia (CLL), Hairy cell leukemia and chronic myeloblastic leukemia. Examples of autoimmune diseases or disorders include, but are not limited to, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE) (including but not limited to lupus nephritis, cutaneous lupus); diabetes mellitus (e.g. Type I diabetes mellitus or insulin dependent diabetes mellitis); multiple sclerosis; Reynaud's syndrome; autoimmune thyroiditis; Hashimoto's thyroiditis; allergic encephalomyelitis; Sjogren's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia etc.

The terms “cell proliferative disorder” and “proliferative disorder” refer to disorders that are associated with some degree of abnormal cell proliferation.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease; preventing or decreasing inflammation and/or tissue/organ damage, decreasing the rate of disease progression, amelioration or palliation of the disease state, and remission or improved prognosis. In some embodiments, antibodies of the invention are used to delay development of a disease or disorder.

An “effective amount” refers to an amount effective, at dosages and for periods of time necessary, to achieve the desired therapeutic or prophylactic result.

A “therapeutically effective amount” of a substance/molecule of the invention may vary according to factors such as the disease state, age, sex, and weight of the individual, and the ability of the substance/molecule, to elicit a desired response in the individual. A therapeutically effective amount is also one in which any toxic or detrimental effects of the substance/molecule are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount would be less than the therapeutically effective amount.

The term “cytotoxic agent” as used herein refers to a substance that inhibits or prevents the function of cells and/or causes destruction of cells.

B. General Methods

In general, the invention provides anti-IFNAR2 antibodies that are useful for treatment of immune-mediated disorders in which a partial or total blockade of type I interferon activity is desired. In one embodiment, the anti-IFNAR2 antibodies of the invention are used to treat autoimmune disorders, such as those indicated above. In another embodiment, the anti-IFNAR2 antibodies provided herein are used to treat graft rejection or graft versus host disease. The unique properties of the anti-IFNAR2 antibodies of the invention make them particularly useful for effecting target levels of immunosuppression in a patient. For patients requiring acute intervention, the anti-IFNAR2 antibodies provided herein which cause broad spectrum ablation of type I interferon activity can be used to effect the largest possible compromise of an undesired immune response. For patients requiring maintenance immunosuppression, the anti-IFNAR2 antibodies provided herein which block one or more (but not necessarily all) species of type I interferon, or which block different species of Type I interferon to various extents, can be used to effect partial compromise of the patient's immune system in order to reduce the risk of undesirable immune responses while leaving some components of the patient's type I interferon-mediated immunity intact in order to avoid undesirable side effects such as infection.

In another aspect, the anti-IFNAR2 antibodies of the invention find utility as reagents for detection and isolation of IFNAR2, such as detection of IFNAR2 expression in various cell types and tissues, including the determination of IFNAR2 receptor density and distribution in cell populations, and cell sorting based on IFNAR2 expression. In yet another aspect, the present anti-IFNAR2 antibodies are useful for the development of IFNAR2 antagonists with type I interferon blocking activity patterns similar to those of the subject antibodies. For example, anti-IFNAR2 antibodies of the invention can be used to determine and identify other antibodies that have the same IFNAR2 binding characteristics and/or anti-viral blocking capabilities. As a further example, anti-IFNAR2 antibodies of the invention can be used to identify other anti-IFNAR2 antibodies that bind substantially the same epitope(s) of IFNAR2 as the antibodies exemplified herein, including linear and conformational epitopes. The anti-IFNAR2 antibodies of the invention can be used in IFNAR2 signal transduction assays to screen for small molecule antagonists of IFNAR2 which will exhibit similar pharmacological effects in blocking the binding of type I interferons to IFNAR2.

Generation of candidate antibodies can be achieved using routine skills in the art, including those described herein, such as the hybridoma technique and screening of phage displayed libraries of binder molecules. These methods are well-established in the art.

Briefly, the anti-IFNAR2 antibodies of the invention can be made by using combinatorial libraries to screen for synthetic antibody clones with the desired activity or activities. In principle, synthetic antibody clones are selected by screening phage libraries containing phage that display various fragments of antibody variable region (Fv) fused to phage coat protein. Such phage libraries are panned by affinity chromatography against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution. Any of the anti-IFNAR2 antibodies of the invention can be obtained by designing a suitable antigen screening procedure to select for the phage clone of interest followed by construction of a full length anti-IFNAR2 antibody clone using the Fv sequences from the phage clone of interest and suitable constant region (Fc) sequences described in Kabat et al., Sequences of Proteins of Immunological Interest, Fifth Edition, NIH Publication 91-3242, Bethesda Md. (1991), vols. 1-3. See also PCT Pub. WO03/102157, and references cited therein.

In one embodiment, anti-IFNAR2 antibodies of the invention are monoclonal. Also encompassed within the scope of the invention are antibody fragments such as Fab, Fab′, Fab′-SH and F(ab′)₂ fragments, and variations thereof, of the anti-IFNAR2 antibodies provided herein. These antibody fragments can be created by traditional means, such as enzymatic digestion, or may be generated by recombinant techniques. Such antibody fragments may be chimeric, human or humanized. These fragments are useful for the diagnostic and therapeutic purposes set forth herein.

Monoclonal antibodies can be obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

The anti-IFNAR2 monoclonal antibodies of the invention can be made using a variety of methods known in the art, including the hybridoma method first described by Kohler et al., Nature, 256:495 (1975), or alternatively they may be made by recombinant DNA methods (e.g., U.S. Pat. No. 4,816,567).

Vectors, Host Cells and Recombinant Methods

For recombinant production of an antibody of the invention, the nucleic acid encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, preferred host cells are of either prokaryotic or eukaryotic (generally mammalian) origin.

Generating Antibodies Using Prokaryotic Host Cells:

Vector Construction

Polynucleotide sequences encoding polypeptide components of the antibody of the invention can be obtained using standard recombinant techniques. Desired polynucleotide sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as λGEM.TM.-11 may be utilized in making a recombinant vector which can be used to transform susceptible host cells such as E. coli LE392.

The expression vector of the invention may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the β-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) Cell 20: 269) using linkers or adaptors to supply any required restriction sites.

In one aspect of the invention, each cistron within the recombinant vector comprises a secretion signal sequence component that directs translocation of the expressed polypeptides. across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In one embodiment of the invention, the signal sequences used in both cistrons of the expression system are STII signal sequences or variants thereof.

In another aspect, the production of the immunoglobulins according to the invention can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB⁻ strains) provide cytoplasm conditions that are favorable for disulfide bond formation, thereby permitting proper folding and assembly of expressed protein subunits. Proba and Pluckthun Gene, 159:203 (1995).

Antibodies of the invention can also be produced by using an expression system in which the quantitative ratio of expressed polypeptide components can be modulated in order to maximize the yield of secreted and properly assembled antibodies of the invention. Such modulation is accomplished at least in part by simultaneously modulating translational strengths for the polypeptide components.

One technique for modulating translational strength is disclosed in Simmons et al., U.S. Pat. No. 5,840,523. It utilizes variants of the translational initiation region (TIR) within a cistron. For a given TIR, a series of amino acid or nucleic acid sequence variants can be created with a range of translational strengths, thereby providing a convenient means by which to adjust this factor for the desired expression level of the specific chain. TIR variants can be generated by conventional mutagenesis techniques that result in codon changes which can alter the amino acid sequence, although silent changes in the nucleotide sequence are preferred. Alterations in the TIR can include, for example, alterations in the number or spacing of Shine-Dalgarno sequences, along with alterations in the signal sequence. One method for generating mutant signal sequences is the generation of a “codon bank” at the beginning of a coding sequence that does not change the amino acid sequence of the signal sequence (i.e., the changes are silent). This can be accomplished by changing the third nucleotide position of each codon; additionally, some amino acids, such as leucine, serine, and arginine, have multiple first and second positions that can add complexity in making the bank. This method of mutagenesis is described in detail in Yansura et al. (1992) METHODS: A Companion to Methods in Enzymol. 4:151-158.

Preferably, a set of vectors is generated with a range of TIR strengths for each cistron therein. This limited set provides a comparison of expression levels of each chain as well as the yield of the desired antibody products under various TIR strength combinations. TIR strengths can be determined by quantifying the expression level of a reporter gene as described in detail in Simmons et al. U.S. Pat. No. 5,840,523. Based on the translational strength comparison, the desired individual TIRs are selected to be combined in the expression vector constructs of the invention.

Prokaryotic host cells suitable for expressing antibodies of the invention include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In one embodiment, gram-negative cells are used. In one embodiment, E. coli cells are used as hosts for the invention. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 ΔfhuA (ΔtonA) ptr3 lac Iq lacL8 ΔompTΔ(nmpc-fepE) degP41 kan^{R} (U.S. Pat. No. 5,639,635). Other strains and derivatives thereof, such as E. coli 294 (ATCC 31,446), E. coli B, E. coli_{γ} 1776 (ATCC 31,537) and E. coli RV308(ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. Typically the host cell should secrete minimal amounts of proteolytic enzymes, and additional protease inhibitors may desirably be incorporated in the cell culture.

Antibody Production

Host cells are transformed with the above-described expression vectors and cultured in conventional nutrient media modified as appropriate for inducing promoters, selecting transformants, or amplifying the genes encoding the desired sequences.

Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the polypeptides of the invention are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., preferably from about 25° C. to about 37° C., preferably at about 30° C. The pH of the medium may be any pH ranging from about 5 to about 9, depending mainly on the host organism. For E. coli, the pH can be from about 6.8 to about 7.4, or about 7.0.

If an inducible promoter is used in the expression vector of the invention, protein expression is induced under conditions suitable for the activation of the promoter. In one aspect of the invention, PhoA promoters are used for controlling transcription of the polypeptides. Accordingly, the transformed host cells are cultured in a phosphate-limiting medium for induction. In one embodiment, the phosphate-limiting medium is the C.R.A.P medium (see, e.g., Simmons et al., J. Immunol. Methods (2002), 263:133-147). A variety of other inducers may be used, according to the vector construct employed, as is known in the art.

In one embodiment, the expressed polypeptides of the present invention are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

In one aspect of the invention, antibody production is conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (the preferred carbon/energy source). Small scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD₅₅₀ of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the polypeptides of the invention, various fermentation conditions can be modified. For example, to improve the proper assembly and folding of the secreted antibody polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J Bio Chem 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210.

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; Georgiou et al., U.S. Pat. No. 5,264,365; Georgiou et al., U.S. Pat. No. 5,508,192; Hara et al., Microbial Drug Resistance, 2:63-72 (1996).

In one embodiment, E. coli strains deficient for proteolytic enzymes and transformed with plasmids overexpressing one or more chaperone proteins are used as host cells in the expression system of the invention.

Antibody Purification

In one embodiment, the antibody protein produced herein is further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In one aspect, Protein A immobilized on a solid phase is used for immunoaffinity purification of the antibody products of the invention. Protein A is a 41kD cell wall protein from Staphylococcus aureas which binds with a high affinity to the Fc region of antibodies. Lindmark et al (1983) J. Immunol. Meth. 62:1-13. The solid phase to which Protein A is immobilized can be a column comprising a glass or silica surface, or a controlled pore glass column or a silicic acid column. In some applications, the column is coated with a reagent, such as glycerol, to possibly prevent nonspecific adherence of contaminants.

As the first step of purification, the preparation derived from the cell culture as described above can be applied onto a Protein A immobilized solid phase to allow specific binding of the antibody of interest to Protein A. The solid phase would then washed to remove contaminants non-specifically bound to the solid phase. Finally the antibody of interest is recovered from the solid phase by elution.

Generating Antibodies Using Eukaryotic Host Cells:

The vector components generally include, but are not limited to, one or more of the following: a signal sequence, an origin of replication, one or more marker genes, an enhancer element, a promoter, and a transcription termination sequence.

(i) Signal Sequence Component

A vector for use in a eukaryotic host cell may also contain a signal sequence or other polypeptide having a specific cleavage site at the N-terminus of the mature protein or polypeptide of interest. The heterologous signal sequence selected generally is one that is recognized and processed (i.e., cleaved by a signal peptidase) by the host cell. In mammalian cell expression, mammalian signal sequences as well as viral secretory leaders, for example, the herpes simplex gD signal, are available.

The DNA for such precursor region is ligated in reading frame to DNA encoding the antibody.

(ii) Origin of Replication

Generally, an origin of replication component is not needed for mammalian expression vectors. For example, the SV40 origin may typically be used only because it contains the early promoter.

(iii) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, where relevant, or (c) supply critical nutrients not available from complex media.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the antibody nucleic acid, such as DHFR, thymidine kinase, metallothionein-I and -II (e.g., primate metallothionein genes), adenosine deaminase, ornithine decarboxylase, etc.

For example, cells transformed with the DHFR selection gene may first be identified by culturing all of the transformants in a culture medium that contains methotrexate (Mtx), a competitive antagonist of DHFR. Appropriate host cells when wild-type DHFR is employed include, for example, the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding an antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

(iv) Promoter Component

Expression and cloning vectors usually contain a promoter that is recognized by the host organism and is operably linked to nucleic acid encoding a polypeptide of interest (e.g., an antibody). Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Antibody polypeptide transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, or from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the Rous Sarcoma Virus long terminal repeat can be used as the promoter.

(v) Enhancer Element Component

Transcription of DNA encoding an antibody polypeptide of the invention by higher eukaryotes can often be increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the antibody polypeptide-encoding sequence, but is generally located at a site 5′ from the promoter.

(vi) Transcription Termination Component

Expression vectors used in eukaryotic host cells will typically also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding an antibody. One useful transcription termination component is the bovine growth hormone polyadenylation region. See WO94/11026 and the expression vector disclosed therein.

(vii) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7,ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR(CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

(viii) Culturing the Host Cells

The host cells used to produce an antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

(ix) Purification of Antibody

When using recombinant techniques, the antibody can be produced intracellularly, or directly secreted into the medium. If the antibody is produced intracellularly, as a first step, the particulate debris, either host cells or lysed fragments, are generally removed, for example, by centrifugation or ultrafiltration. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being a generally acceptable purification technique. The suitability of affinity reagents such as protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to further purification steps, as necessary, for example by low pH hydrophobic interaction chromatography using an elution buffer at a pH between about 2.5-4.5, generally performed at low salt concentrations (e.g., from about 0-0.25 M salt).

It should be noted that, in general, techniques and methodologies for preparing antibodies for use in research, testing and clinical use are well-established in the art, consistent with the above and/or as deemed appropriate by one skilled in the art for the particular antibody of interest.

Activity Assays

Antibodies of the invention can be characterized for their physical/chemical properties and biological functions by various assays known in the art.

Purified antibodies can be further characterized by a series of assays including, but not limited to, N-terminal sequencing, amino acid analysis, non-denaturing size exclusion high pressure liquid chromatography (HPLC), mass spectrometry, ion exchange chromatography and papain digestion.

Where necessary, antibodies are analyzed for their biological activity. In some embodiments, antibodies of the invention are tested for their antigen binding activity. The antigen binding assays that are known in the art and can be used herein include without limitation any direct or competitive binding assays using techniques such as western blots, radioimmunoassays, ELISA (enzyme linked immunosorbent assay), “sandwich” immunoassays, immunoprecipitation assays, fluorescent immunoassays, and protein A immunoassays.

In one embodiment, the invention contemplates an altered antibody that possesses some but not all effector functions, which make it a desirable candidate for many applications in which the half life of the antibody in vivo is important yet certain effector functions (such as complement and ADCC) are unnecessary or deleterious. In certain embodiments, the Fc activities of the antibody are measured to ensure that only the desired properties are maintained. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). An example of an in vitro assay to assess ADCC activity of a molecule of interest is described in U.S. Pat. No. 5,500,362 or U.S. Pat. No. 5,821,337. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS(USA) 95:652-656 (1998). C1q binding assays may also be carried out to confirm that the antibody is unable to bind C1q and hence lacks CDC activity. To assess complement activation, a CDC assay, e.g. as described in Gazzano-Santoro et al., J. Immunol. Methods 202:163 (1996), may be performed. FcRn binding and in vivo clearance/half life determinations can also be performed using methods known in the art.

Humanized Antibodies

The invention encompasses humanized antibodies. Various methods for humanizing non-human antibodies are known in the art. For example, a humanized antibody can have one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al. (1986) Nature 321:522-525; Riechmann et al. (1988) Nature 332:323-327; Verhoeyen et al. (1988) Science 239:1534-1536), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies can be important to reduce antigenicity. According to the so-called “best-fit” method, the sequence of the variable domain of a rodent antibody is screened against the entire library of known human variable-domain sequences. The human sequence which is closest to that of the rodent is then accepted as the human framework for the humanized antibody (Sims et al. (1993) J. Immunol. 151:2296; Chothia et al. (1987) J. Mol. Biol. 196:901. Another method uses a particular framework derived from the consensus sequence of all human antibodies of a particular subgroup of light or heavy chains. The same framework may be used for several different humanized antibodies (Carter et al. (1992) Proc. Natl. Acad. Sci. USA, 89:4285; Presta et al. (1993) J. Immunol., 151:2623.

It is further generally desirable that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Antibody Variants

In one aspect, the invention provides antibodies comprising modifications in the interface of Fc polypeptides comprising the Fc region, wherein the modifications facilitate and/or promote heterodimerization. These modifications comprise introduction of a protuberance into a first Fc polypeptide and a cavity into a second Fc polypeptide, wherein the protuberance is positionable in the cavity so as to promote complexing of the first and second Fc polypeptides. Methods of generating antibodies with these modifications are known in the art, e.g., as described in U.S. Pat. No. 5,731,168.

In some embodiments, amino acid sequence modification(s) of the antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution can be made to arrive at the final construct, provided that the final construct possesses the desired characteristics. The amino acid alterations may be introduced in the subject antibody amino acid sequence at the time that sequence is made.

A useful method for identification of certain residues or regions of the antibody that are preferred locations for mutagenesis is called “alanine scanning mutagenesis” as described by Cunningham and Wells (1989) Science, 244:1081-1085. Here, a residue or group of target residues are identified (e.g., charged residues such as arg, asp, his, lys, and glu) and replaced by a neutral or negatively charged amino acid (e.g., alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed immunoglobulins are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme (e.g. for ADEPT) or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in Table A under the heading of “preferred substitutions”. If such substitutions result in a change in biological activity, then more substantial changes, denominated “exemplary substitutions” in Table A, or as further described below in reference to amino acid classes, may be introduced and the products screened.

TABLE A

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (1), Pro (P), Phe (F), Trp (W), Met (M)

(2) uncharged polar: Gly (G), Ser (S), Thr (T), Cys (C), Tyr (Y), Asn (N), Gln (Q)

(3) acidic: Asp (D), Glu (E)

(4) basic; Lys (K), Arg (R), His(H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class. Such substituted residues also may be introduced into the conservative substitution sites or, more preferably, into the remaining (non-conserved) sites.

One type of substitutional variant involves substituting one or more hypervariable region residues of a parent antibody (e.g. a humanized or human antibody). Generally, the resulting variant(s) selected for further development will have modified (e.g., improved) biological properties relative to the parent antibody from which they are generated. A convenient way for generating such substitutional variants involves affinity maturation using phage display. Briefly, several hypervariable region sites (e.g. 6-7 sites) are mutated to generate all possible amino acid substitutions at each site. The antibodies thus generated are displayed from filamentous phage particles as fusions to at least part of a phage coat protein (e.g., the gene III product of M13) packaged within each particle. The phage-displayed variants are then screened for their biological activity (e.g. binding affinity) as herein disclosed. In order to identify candidate hypervariable region sites for modification, scanning mutagenesis (e.g., alanine scanning) can be performed to identify hypervariable region residues contributing significantly to antigen binding. Alternatively, or additionally, it may be beneficial to analyze a crystal structure of the antigen-antibody complex to identify contact points between the antibody and antigen. Such contact residues and neighboring residues are candidates for substitution according to techniques known in the art, including those elaborated herein. Once such variants are generated, the panel of variants is subjected to screening using techniques known in the art, including those described herein, and antibodies with superior properties in one or more relevant assays may be selected for further development.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

It may be desirable to introduce one or more amino acid modifications in an Fc region of antibodies of the invention, thereby generating a Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions including that of a hinge cysteine.

In accordance with this description and the teachings of the art, it is contemplated that in some embodiments, an antibody of the invention may comprise one or more alterations as compared to the wild type counterpart antibody, e.g. in the Fc region. These antibodies would nonetheless retain substantially the same characteristics required for therapeutic utility as compared to their wild type counterpart. For example, it is thought that certain alterations can be made in the Fc region that would result in altered (i.e., either improved or diminished) C1q binding and/or Complement Dependent Cytotoxicity (CDC), e.g., as described in WO99/51642. See also Duncan & Winter Nature 322:738-40 (1988); U.S. Pat. No. 5,648,260; U.S. Pat. No. 5,624,821; and WO94/29351 concerning other examples of Fc region variants.

Immunoconjugates

In another aspect, the invention provides immunoconjugates, or antibody-drug conjugates (ADC), comprising an antibody conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate).

The use of antibody-drug conjugates for the local delivery of cytotoxic or cytostatic agents, i.e. drugs to kill or inhibit tumor cells in the treatment of cancer (Syrigos and Epenetos (1999) Anticancer Research 19:605-614; Niculescu-Duvaz and Springer (1997) Adv. Drg Del. Rev. 26:151-172; U.S. Pat. No. 4,975,278) allows targeted delivery of the drug moiety to tumors, and intracellular accumulation therein, where systemic administration of these unconjugated drug agents may result in unacceptable levels of toxicity to normal cells as well as the tumor cells sought to be eliminated (Baldwin et al., (1986) Lancet pp. (Mar. 15, 1986):603-05; Thorpe, (1985) “Antibody Carriers Of Cytotoxic Agents In Cancer Therapy: A Review,” in Monoclonal Antibodies '84: Biological And Clinical Applications, A. Pinchera et al. (ed.s), pp. 475-506). Maximal efficacy with minimal toxicity is sought thereby. Both polyclonal antibodies and monoclonal antibodies have been reported as useful in these strategies (Rowland et al., (1986) Cancer Immunol. Immunother., 21:183-87). Drugs used in these methods include daunomycin, doxorubicin, methotrexate, and vindesine (Rowland et al., (1986) supra). Toxins used in antibody-toxin conjugates include bacterial toxins such as diphtheria toxin, plant toxins such as ricin, small molecule toxins such as geldanamycin (Mandler et al (2000) Jour. of the Nat. Cancer Inst. 92(19):1573-1581; Mandler et al (2000) Bioorganic & Med. Chem. Letters 10: 1025-1028; Mandler et al (2002) Bioconjugate Chem. 13:786-791), maytansinoids (EP 1391213; Liu et al., (1996) Proc. Natl. Acad. Sci. USA 93:8618-8623), and calicheamicin (Lode et al (1998) Cancer Res. 58:2928; Hinman et al (1993) Cancer Res. 53:3336-3342). The toxins may effect their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

ZEVALIN® (ibritumomab tiuxetan, Biogen/Idec) is an antibody-radioisotope conjugate composed of a murine IgG1 kappa monoclonal antibody directed against the CD20 antigen found on the surface of normal and malignant B lymphocytes and ¹¹¹In or ⁹⁰Y radioisotope bound by a thiourea linker-chelator (Wiseman et al (2000) Eur. Jour. Nucl. Med. 27(7):766-77; Wiseman et al (2002) Blood 99(12):4336-42; Witzig et al (2002) J. Clin. Oncol. 20(10):2453-63; Witzig et al (2002) J. Clin. Oncol. 20(15):3262-69). Although ZEVALIN has activity against B-cell non-Hodgkin's Lymphoma (NHL), administration results in severe and prolonged cytopenias in most patients. MYLOTARG™ (gemtuzumab ozogamicin, Wyeth Pharmaceuticals), an antibody drug conjugate composed of a hu CD33 antibody linked to calicheamicin, was approved in 2000 for the treatment of acute myeloid leukemia by injection (Drugs of the Future (2000) 25(7):686; U.S. Pat. Nos. 4,970,198; 5,079,233; 5,585,089; 5,606,040; 5,693,762; 5,739,116; 5,767,285; 5,773,001). Cantuzumab mertansine (Immunogen, Inc.), an antibody drug conjugate composed of the huC242 antibody linked via the disulfide linker SPP to the maytansinoid drug moiety, DM1, is tested for the treatment of cancers that express CanAg, such as colon, pancreatic, gastric, and others. MLN-2704 (Millennium Pharm., BZL Biologics, Immunogen Inc.), an antibody drug conjugate composed of the anti-prostate specific membrane antigen (PSMA) monoclonal antibody linked to the maytansinoid drug moiety, DM1, is tested for the potential treatment of prostate tumors. The auristatin peptides, auristatin E (AE) and monomethylauristatin (MMAE), synthetic analogs of dolastatin, were conjugated to chimeric monoclonal antibodies cBR96 (specific to Lewis Y on carcinomas) and cAC10 (specific to CD30 on hematological malignancies) (Doronina et al (2003) Nature Biotechnology 21(7):778-784) and are under therapeutic development.

Chemotherapeutic agents useful in the generation of immunoconjugates are described herein (above). Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. See, e.g., WO 93/21232 published Oct. 28, 1993. A variety of radionuclides are available for the production of radioconjugated antibodies. Examples include ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

Conjugates of an antibody and one or more small molecule toxins, such as a calicheamicin, maytansinoids, dolostatins, aurostatins, a trichothecene, and CC1065, and the derivatives of these toxins that have toxin activity, are also contemplated herein.

Maytansine and Maytansinoids

In some embodiments, the immunoconjugate comprises an antibody of the invention conjugated to one or more maytansinoid molecules.

Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

Maytansinoid drug moieties are attractive drug moieties in antibody drug conjugates because they are: (i) relatively accessible to prepare by fermentation or chemical modification, derivatization of fermentation products, (ii) amenable to derivatization with functional groups suitable for conjugation through the non-disulfide linkers to antibodies, (iii) stable in plasma, and (iv) effective against a variety of tumor cell lines.

Exemplary embodiments of maytansinoid drug moieities include: DM1; DM3; and DM4. Immunoconjugates containing maytansinoids, methods of making same, and their therapeutic use are disclosed, for example, in U.S. Pat. Nos. 5,208,020, 5,416,064 and European Patent EP 0 425 235 B1, the disclosures of which are hereby expressly incorporated by reference. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay. Chari et al., Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA.1 that binds the HER-2/neu oncogene. The cytotoxicity of the TA.1-maytansonoid conjugate was tested in vitro on the human breast cancer cell line SK-BR-3, which expresses 3×10⁵ HER-2 surface antigens per cell. The drug conjugate achieved a degree of cytotoxicity similar to the free maytansinoid drug, which could be increased by increasing the number of maytansinoid molecules per antibody molecule. The A7-maytansinoid conjugate showed low systemic cytotoxicity in mice.

Antibody-maytansinoid conjugates can be prepared by chemically linking an antibody to a maytansinoid molecule without significantly diminishing the biological activity of either the antibody or the maytansinoid molecule. See, e.g., U.S. Pat. No. 5,208,020 (the disclosure of which is hereby expressly incorporated by reference). An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. Preferred maytansinoids are maytansinol and maytansinol analogues modified in the aromatic ring or at other positions of the maytansinol molecule, such as various maytansinol esters.

There are many linking groups known in the art for making antibody-maytansinoid conjugates, including, for example, those disclosed in U.S. Pat. No. 5,208,020 or EP Patent 0 425 235 B1, Chari et al., Cancer Research 52:127-131 (1992), and U.S. patent application Ser. No. 10/960,602, filed Oct. 8, 2004, the disclosures of which are hereby expressly incorporated by reference. Antibody-maytansinoid conjugates comprising the linker component SMCC may be prepared as disclosed in U.S. patent application Ser. No. 10/960,602, filed Oct. 8, 2004. The linking groups include disulfide groups, thioether groups, acid labile groups, photolabile groups, peptidase labile groups, or esterase labile groups, as disclosed in the above-identified patents, disulfide and thioether groups being preferred. Additional linking groups are described and exemplified herein.

Conjugates of the antibody and maytansinoid may be made using a variety of bifunctional protein coupling agents such as N-succinimidyl-3-(2-pyridyldithio)propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particularly preferred coupling agents include N-succinimidyl-3-(2-pyridyldithio)propionate (SPDP) (Carlsson et al., Biochem. J. 173:723-737 (1978)) and N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hydroxymethyl, the C-15 position modified with a hydroxyl group, and the C-20 position having a hydroxyl group. In a preferred embodiment, the linkage is formed at the C-3 position of maytansinol or a maytansinol analogue.

Auristatins and Dolostatins

In some embodiments, the immunoconjugate comprises an antibody of the invention conjugated to dolastatins or dolostatin peptidic analogs and derivatives, the auristatins (U.S. Pat. Nos. 5,635,483; 5,780,588). Dolastatins and auristatins have been shown to interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). The dolastatin or auristatin drug moiety may be attached to the antibody through the N (amino) terminus or the C (carboxyl) terminus of the peptidic drug moiety (WO 02/088172).

Exemplary auristatin embodiments include the N-terminus linked monomethylauristatin drug moieties DE and DF, disclosed in “Monomethylvaline Compounds Capable of Conjugation to Ligands”, U.S. Ser. No. 10/983,340, filed Nov. 5, 2004, the disclosure of which is expressly incorporated by reference in its entirety.

An exemplary auristatin embodiments are MMAE and MMAF. Additional exemplary embodiments comprising MMAE or MMAF and various linker components (described further herein) Ab-MC-vc-PAB-MMAF, Ab-MC-vc-PAB-MMAE, Ab-MC-MMAE and Ab-MC-MMAF.

Typically, peptide-based drug moieties can be prepared by forming a peptide bond between two or more amino acids and/or peptide fragments. Such peptide bonds can be prepared, for example, according to the liquid phase synthesis method (see E. Schröder and K. Lübke, “The Peptides”, volume 1, pp 76-136, 1965, Academic Press) that is well known in the field of peptide chemistry. The auristatin/dolastatin drug moieties may be prepared according to the methods of: U.S. Pat. No. 5,635,483; U.S. Pat. No. 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; and Pettit et al (1996) J. Chem. Soc. Perkin Trans. 1 5:859-863. See also Doronina (2003) Nat Biotechnol 21(7):778-784; “Monomethylvaline Compounds Capable of Conjugation to Ligands”, U.S. Ser. No. 10/983,340, filed Nov. 5, 2004, hereby incorporated by reference in its entirety (disclosing, e.g., linkers and methods of preparing monomethylvaline compounds such as MMAE and MMAF conjugated to linkers).

Calicheamicin

In other embodiments, the immunoconjugate comprises an antibody of the invention conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ₁^{I}, α₂^{I}, α₃^{I}, N-acetyl-γ₁^{I}, PSAG and θ^{I}₁(Hinman et al., Cancer Research 53:3336-3342 (1993), Lode et al., Cancer Research 58:2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

Other Cytotoxic Agents

Other antitumor agents that can be conjugated to the antibodies of the invention include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates an immunoconjugate formed between an antibody and a compound with nucleolytic activity (e.g., a ribonuclease or a DNA endonuclease such as a deoxyribonuclease; DNase).

For selective destruction of the tumor, the antibody may comprise a highly radioactive atom. A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the conjugate is used for detection, it may comprise a radioactive atom for scintigraphic studies, for example tc^{99m} or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc^{99m} or I¹²³, Re¹⁸⁶, Re¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes other methods in detail.

Conjugates of the antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

The compounds of the invention expressly contemplate, but are not limited to, ADC prepared with cross-linker reagents: BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A). See pages 467-498, 2003-2004 Applications Handbook and Catalog.

Preparation of Antibody Drug Conjugates

In the antibody drug conjugates (ADC) of the invention, an antibody (Ab) is conjugated to one or more drug moieties (D), e.g. about 1 to about 20 drug moieties per antibody, through a linker (L). The ADC of Formula I may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. Additional methods for preparing ADC are described herein.
Ab-(L-D)ₚ I

The linker may be composed of one or more linker components. Exemplary linker components include 6-maleimidocaproyl (“MC”), maleimidopropanoyl (“MP”), valine-citrulline (“val-cit”), alanine-phenylalanine (“ala-phe”), p-aminobenzyloxycarbonyl (“PAB”), N-Succinimidyl 4-(2-pyridylthio) pentanoate (“SPP”), N-Succinimidyl 4-(N-maleimidomethyl) cyclohexane-1 carboxylate (“SMCC”), and N-Succinimidyl (4-iodo-acetyl) aminobenzoate (“SIAB”). Additional linker components are known in the art and some are described herein. See also “Monomethylvaline Compounds Capable of Conjugation to Ligands”, U.S. Ser. No. 10/983,340, filed Nov. 5, 2004, the contents of which are hereby incorporated by reference in its entirety.

In some embodiments, the linker may comprise amino acid residues. Exemplary amino acid linker components include a dipeptide, a tripeptide, a tetrapeptide or a pentapeptide. Exemplary dipeptides include: valine-citrulline (vc or val-cit), alanine-phenylalanine (af or ala-phe). Exemplary tripeptides include: glycine-valine-citrulline (gly-val-cit) and glycine-glycine-glycine (gly-gly-gly). Amino acid residues which comprise an amino acid linker component include those occurring naturally, as well as minor amino acids and non-naturally occurring amino acid analogs, such as citrulline. Amino acid linker components can be designed and optimized in their selectivity for enzymatic cleavage by a particular enzymes, for example, a tumor-associated protease, cathepsin B, C and D, or a plasmin protease.

Exemplary linker component structures are shown below (wherein the wavy line indicates sites of covalent attachment to other components of the ADC):

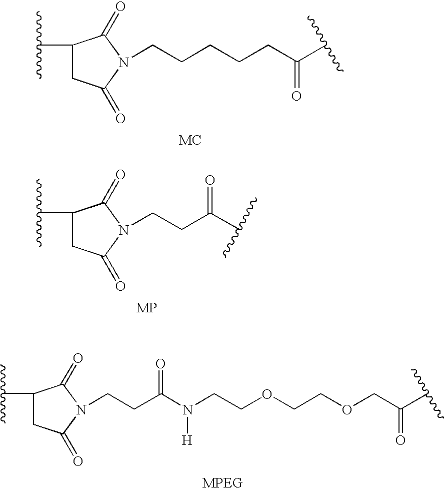

Additional exemplary linker components and abbreviations include wherein the antibody (Ab) and linker are depicted, and p is 1 to about 8):

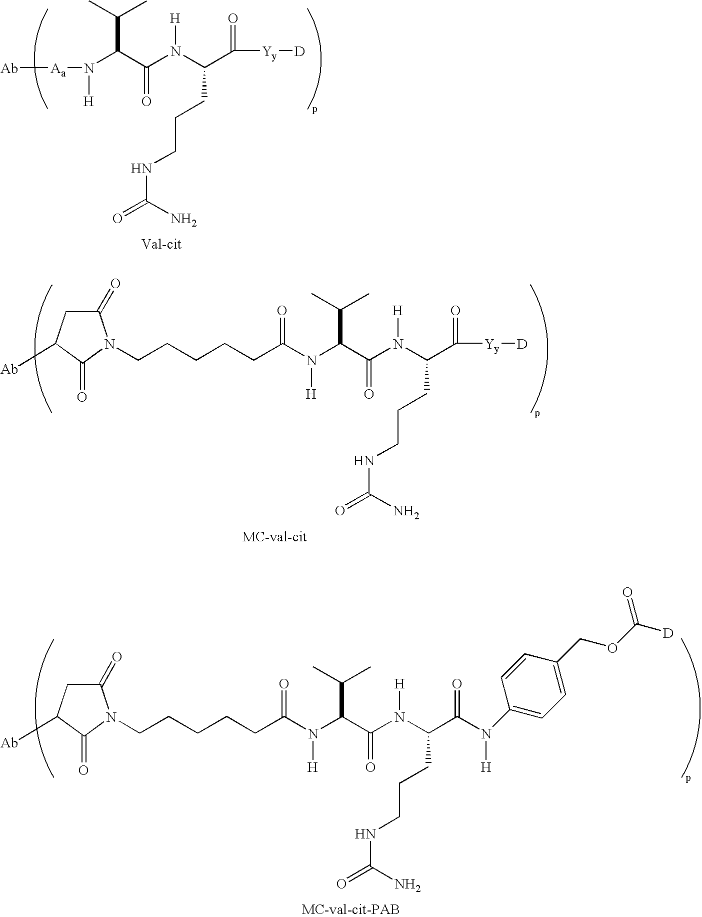

Nucleophilic groups on antibodies include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups. Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by introducing one, two, three, four, or more cysteine residues (e.g., preparing mutant antibodies comprising one or more non-native cysteine amino acid residues).

Antibody drug conjugates of the invention may also be produced by modification of the antibody to introduce electrophilic moieties, which can react with nucleophilic subsituents on the linker reagent or drug. The sugars of glycosylated antibodies may be oxidized, e.g. with periodate oxidizing reagents, to form aldehyde or ketone groups which may react with the amine group of linker reagents or drug moieties. The resulting imine Schiff base groups may form a stable linkage, or may be reduced, e.g. by borohydride reagents to form stable amine linkages. In one embodiment, reaction of the carbohydrate portion of a glycosylated antibody with either glactose oxidase or sodium meta-periodate may yield carbonyl (aldehyde and ketone) groups in the protein that can react with appropriate groups on the drug (Hermanson, Bioconjugate Techniques). In another embodiment, proteins containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such aldehyde can be reacted with a drug moiety or linker nucleophile.

Likewise, nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

In yet another embodiment, the antibody may be conjugated to a “receptor” (such streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

Antibody (Ab)-MC-MMAE may be prepared by conjugation of any of the antibodies provided herein with MC-MMAE as follows. Antibody, dissolved in 500 mM sodium borate and 500 mM sodium chloride at pH 8.0 is treated with an excess of 100 mM dithiothreitol (DTT). After incubation at 37° C. for about 30 minutes, the buffer is exchanged by elution over Sephadex G25 resin and eluted with PBS with 1 mM DTPA. The thiol/Ab value is checked by determining the reduced antibody concentration from the absorbance at 280 nm of the solution and the thiol concentration by reaction with DTNB (Aldrich, Milwaukee, Wis.) and determination of the absorbance at 412 nm. The reduced antibody dissolved in PBS is chilled on ice. The drug linker reagent, maleimidocaproyl-monomethyl auristatin E (MMAE), i.e. MC-MMAE, dissolved in DMSO, is diluted in acetonitrile and water at known concentration, and added to the chilled reduced antibody 2H9 in PBS. After about one hour, an excess of maleimide is added to quench the reaction and cap any unreacted antibody thiol groups. The reaction mixture is concentrated by centrifugal ultrafiltration and 2H9-MC-MMAE is purified and desalted by elution through G25 resin in PBS, filtered through 0.2 μm filters under sterile conditions, and frozen for storage.

Antibody-MC-MMAF may be prepared by conjugation of any of the antibodies provided herein with MC-MMAF following the protocol provided for preparation of Ab-MC-MMAE.

Antibody-MC-val-cit-PAB-MMAE is prepared by conjugation of any of the antibodies provided herein with MC-val-cit-PAB-MMAE following the protocol provided for preparation of Ab-MC-MMAE.

Antibody-MC-val-cit-PAB-MMAF is prepared by conjugation of any of the antibodies provided herein with MC-val-cit-PAB-MMAF following the protocol provided for preparation of Ab-MC-MMAE.

Antibody-SMCC-DM1 is prepared by conjugation of any of the antibodies provided herein with SMCC-DM1 as follows. Purified antibody is derivatized with (Succinimidyl 4-(N-maleimidomethyl) cyclohexane-1-carboxylate (SMCC, Pierce Biotechnology, Inc) to introduce the SMCC linker. Specifically, antibody is treated at 20 mg/mL in 50 mM potassium phosphate/50 mM sodium chloride/2 mM EDTA, pH 6.5 with 7.5 molar equivalents of SMCC (20 mM in DMSO, 6.7 mg/mL). After stirring for 2 hours under argon at ambient temperature, the reaction mixture is filtered through a Sephadex G25 column equilibrated with 50 mM potassium phosphate/50 mM sodium chloride/2 mM EDTA, pH 6.5. Antibody containing fractions are pooled and assayed.

Antibody-SMCC prepared thus is diluted with 50 mM potassium phosphate/50 mM sodium chloride/2 mM EDTA, pH 6.5, to a final concentration of about 10 mg/ml, and reacted with a 10 mM solution of DM1 in dimethylacetamide. The reaction is stirred at ambient temperature under argon 16.5 hours. The conjugation reaction mixture is filtered through a Sephadex G25 gel filtration column (1.5×4.9 cm) with 1×PBS at pH 6.5. The DM1 drug to antibody ratio (p) may be about 2 to 5, as measured by the absorbance at 252 nm and at 280 nm.

Ab-SPP-DM1 is prepared by conjugation of any of the antibodies provided herein with SPP-DM1 as follows. Purified antibody is derivatized with N-succinimidyl-4-(2-pyridylthio)pentanoate to introduce dithiopyridyl groups. Antibody (376.0 mg, 8 mg/mL) in 44.7 mL of 50 mM potassium phosphate buffer (pH 6.5) containing NaCl (50 mM) and EDTA (1 mM) is treated with SPP (5.3 molar equivalents in 2.3 mL ethanol). After incubation for 90 minutes under argon at ambient temperature, the reaction mixture is gel filtered through a ephadex G25 column equilibrated with 35 mM sodium citrate, 154 mM NaCl, 2 mM EDTA. Antibody containing fractions were pooled and assayed. The degree of modification of the antibody is determined as described above.

Antibody-SPP-Py (about 10 μmoles of releasable 2-thiopyridine groups) is diluted with the above 35 mM sodium citrate buffer, pH 6.5, to a final concentration of about 2.5 mg/mL. DM1 (1.7 equivalents, 17 μmoles) in 3.0 mM dimethylacetamide (DMA, 3% v/v in the final reaction mixture) is then added to the antibody solution. The reaction proceeds at ambient temperature under argon for about 20 hours. The reaction is loaded on a Sephacryl S300 gel filtration column (5.0 cm×90.0 cm, 1.77 L) equilibrated with 35 mM sodium citrate, 154 mM NaCl, pH 6.5. The flow rate may be about 5.0 mL/min and 65 fractions (20.0 mL each) are collected. The number of DM1 drug molecules linked per antibody molecule (p′) is determined by measuring the absorbance at 252 nm and 280 nm, and may be about 2 to 4 DM1 drug moities per 2H9 antibody.

Antibody-BMPEO-DM1 is prepared by conjugation of any of the antibodies provided herein with BMPEO-DM 1 as follows. The antibody is modified by the bis-maleimido reagent BM(PEO)4 (Pierce Chemical), leaving an unreacted maleimido group on the surface of the antibody. This may be accomplished by dissolving BM(PEO)4 in a 50% ethanol/water mixture to a concentration of 10 mM and adding a tenfold molar excess to a solution containing antibody in phosphate buffered saline at a concentration of approximately 1.6 mg/ml (10 micromolar) and allowing it to react for 1 hour to form antibody-linker intermediate, 2H9-BMPEO. Excess BM(PEO)₄ is removed by gel filtration (HiTrap column, Pharmacia) in 30 mM citrate, pH 6 with 150 mM NaCl buffer. An approximate 10 fold molar excess DM1 is dissolved in dimethyl acetamide (DMA) and added to the 2H9-BMPEO intermediate. Dimethyl formamide (DMF) may also be employed to dissolve the drug moiety reagent. The reaction mixture is allowed to react overnight before gel filtration or dialysis into PBS to remove unreacted DM1. Gel filtration on S200 columns in PBS was used to remove high molecular weight aggregates and furnish purified 2H9-BMPEO-DM 1.

Antibody Derivatives

Antibodies of the invention can be further modified to contain additional nonproteinaceous moieties that are known in the art and readily available. In one embodiment, the moieties suitable for derivatization of the antibody are water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymers are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

Pharmaceutical Formulations

Therapeutic formulations comprising an antibody of the invention are prepared for storage by mixing the antibody having the desired degree of purity with optional physiologically acceptable carriers, excipients or stabilizers (Remington's Pharmaceutical Sciences 16th edition, Osol, A. Ed. (1980)), in the form of aqueous solutions, lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the immunoglobulin of the invention, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773;919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated immunoglobulins remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Uses

An antibody of the invention may be used in, for example, in vitro, ex vivo and in vivo therapeutic methods. Antibodies of the invention can be used as an antagonist to partially or fully block the specific antigen activity in vitro, ex vivo and/or in vivo. Moreover, at least some of the antibodies of the invention can neutralize antigen activity from other species. Accordingly, antibodies of the invention can be used to inhibit a specific antigen activity, e.g., in a cell culture containing the antigen, in human subjects or in other mammalian subjects having the antigen with which an antibody of the invention cross-reacts (e.g. chimpanzee, baboon, marmoset, cynomolgus and rhesus, pig or mouse). In one embodiment, an antibody of the invention can be used for inhibiting antigen activities by contacting the antibody with the antigen such that antigen activity is inhibited. In one embodiment, the antigen is a human protein molecule.

In one embodiment, an antibody of the invention can be used in a method for inhibiting an antigen in a subject suffering from a disorder in which the antigen activity is detrimental, comprising administering to the subject an antibody of the invention such that the antigen activity in the subject is inhibited. In one embodiment, the antigen is a human protein molecule and the subject is a human subject. Alternatively, the subject can be a mammal expressing the antigen with which an antibody of the invention binds. Still further the subject can be a mammal into which the antigen has been introduced (e.g., by administration of the antigen or by expression of an antigen transgene). An antibody of the invention can be administered to a human subject for therapeutic purposes. Moreover, an antibody of the invention can be administered to a non-human mammal expressing an antigen with which the antibody cross-reacts (e.g., a primate, pig or mouse) for veterinary purposes or as an animal model of human disease. Regarding the latter, such animal models may be useful for evaluating the therapeutic efficacy of antibodies of the invention (e.g., testing of dosages and time courses of administration). Antibodies of the invention can be used to treat, inhibit, delay progression of, prevent/delay recurrence of, ameliorate, or prevent diseases, disorders or conditions associated with abnormal expression and/or activity of type I interferons/IFNAR2, including but not limited to inflammatory, autoimmune and other immunologic disorders.

In one aspect, a blocking antibody of the invention is specific to IFNAR2, and inhibits IFNAR2 activity by blocking or interfering with the ligand-receptor interaction involving the IFNAR2, thereby inhibiting the corresponding signal pathway and other associated molecular or cellular events. The invention also features receptor-specific antibodies which do not necessarily prevent (or only partially prevent) ligand binding, but significantly interfere with receptor activation, thereby inhibiting any responses that would normally be initiated by the ligand binding.

In certain embodiments, an immunoconjugate comprising an antibody conjugated with a cytotoxic agent is administered to the patient. In some embodiments, the immunoconjugate and/or antigen to which it is bound is/are internalized by the cell, resulting in increased therapeutic efficacy of the immunoconjugate in killing the target cell to which it binds. In one embodiment, the cytotoxic agent targets or interferes with nucleic acid in the target cell. Examples of such cytotoxic agents include any of the chemotherapeutic agents noted herein (such as a maytansinoid or a calicheamicin), a radioactive isotope, or a ribonuclease or a DNA endonuclease.

Antibodies of the invention can be used either alone or in combination with other compositions in a therapy. For instance, an antibody of the invention may be co-administered with another antibody, and/or adjuvant/therapeutic agents (e.g., steroids). For instance, an antibody of the invention may be combined with an anti-inflammatory and/or antiseptic in a treatment scheme, e.g. in treating any of the diseases described herein, including inflammatory, autoimmune and other immunological disorders. Such combined therapies noted above include combined administration (where the two or more agents are included in the same or separate formulations), and separate administration, in which case, administration of the antibody of the invention can occur prior to, and/or following, administration of the adjunct therapy or therapies.

An antibody of the invention (and adjunct therapeutic agent) can be administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. In addition, the antibody is suitably administered by pulse infusion, particularly with declining doses of the antibody. Dosing can be by any suitable route, e.g. by injections, such as intravenous or subcutaneous injections, depending in part on whether the administration is brief or chronic.

The antibody composition of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibodies of the invention present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with other agents such as chemotherapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. An exemplary dosing regimen comprises administering an initial loading dose of about 4 mg/kg, followed by a weekly maintenance dose of about 2 mg/kg of the antibody. However, other dosage regimens may be useful. The progress of this therapy is easily monitored by conventional techniques and assays.

Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, etc. The containers may be formed from a variety of materials such as glass or plastic. The container holds a composition which is by itself or when combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular conditioner. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The following are examples of the methods and compositions of the invention. It is understood that various other embodiments may be practiced, given the general description provided above.

### EXAMPLE

Materials and Methods

Generation of Antibodies to IFNAR2

Antibodies to IFNAR2 were generated essentially as described in US Pub. 2003-0018174, published Jan. 23, 2003.

Determination of the Affinities of mAbs

Biosensor CM5 chips (Biacore cat# BR-1000-14; Neuchatel, Switzerland) were used for the binding affinity assay on a Biacore 3000 instrument. Immobilization of the human interferon antibody receptor 2 (IFNAR2) ECD protein fused to an IgG 1 (IFNAR2.ECD.IgG1), produced in CHO cells, onto the chip was done using the 10 mM sodium acetate pH 4.8 buffer. Antibodies to the receptor beta chain were used as analytes in the assay. Each antibody ranged from 500 nM to 0.69 nM in a series of ⅓ serial dilutions in PBS plus 0.05% Tween20™. The binding was done at 37° C. with a 60 minute dissociation rate for the top two analyte concentrations. Between each injection of an analyte, the chip was regenerated with two injections of 20 mM hydrochloric acid. The KD was measured by fitting the kinetics ka/kd simultaneously using the 1:1 (Langmuir) binding model.

Anti-Viral Assay

Experiments consistent with the following protocol were performed to measure interferon-mediated anti-viral activity and ability of various antibodies to neutralize this activity. Cell lines sensitive to ECMV (e.g., WISH cell line) are killed by the virus in tissue culture. If interferon (IFN) is present during the incubation with ECMV, the cells are protected from killing by the virus. Neutralization activity of anti-IFN receptor antibodies can be assessed by adding the antibodies with interferon into the tissue cultures. Neutralization activity of an antibody could be determined based on its capacity to block the protective activity of interferon.

Materials: (all volume calculations below correspond to preparations for total final volume of 1 L)

WISH fibroblasts (human) FIBROBLAST MEDIA

| | | | |
|---|---|---|---|
| Media: | Eagle's MEM (ATCC#30-2003) | 900 | ml |
| | (already contains non-essential | | |
| | amino acids, 2 mM L-glu, 1 mM | | |
| | sodium pyruvate, and 1500 mg/L | | |
| | Na bicarbonate) | | |
| | 10% fetal calf serum (FCS) | 100 | ml |
| | (heat inactivated) | | |
| | 10 mM HEPES | 10 | ml (1M) |
| | 1× Penicillin/Streptomycin | 10 | ml |
| Media D | DMEM (high glu) | 900 | ml |
| | 10% FCS (HI) | 100 | ml |
| | 0.4% Sodium bicarbonate | 53 | ml (7.5%) |
| | 4 mM HEPES | 4 | ml (1M) |
| | 40 mM L-glutamine | 20 | ml (200 mM) |
| | Penicillin/Streptomycin (1×) | 10 | ml (10×) |
| Bioassay | DMEM (high glu) | 900 | ml |
| Media | 2% FCS (HI) | 100 | ml |
| | 0.4% Sodium bicarbonate | 53 | ml (7.5%) |
| | 4 mM HEPES | 4 | ml (1M) |
| | 40 mM L-glutamine | 20 | ml (200 mM) |
| | Penicillin/Streptomycin (1×) | 10 | ml (10×) |

EMC virus (mouse encephalomyocarditis, ATCC VR-129B), stored at −80° C.; stored in 1 ml aliquots (titer is ˜3.25×10⁸ PFU/ml).
Crystal Violet solution (0.5%)
96-well flat-bottomed plates
12-well mulitwell plate washer manifold/vacuum filter system
Procedures:
Fibroblasts were cultured in FIBROBLAST media.
Day 1:
1. Fibroblasts were seeded in 96-well plates at 2×10⁵ cells/ml (100 μl/well) in Media D and incubated at 37° C., 5% CO₂ for 18-24 h.
   Day 2:
1. Dilutions of IFN (e.g., IFN-α (either produced in-house at Genentech, Inc. or purchased from PBL (Piscataway, N.J.)) and leukocyte interferon (Sigma®; St. Louis, Mo.)) in media D were performed with or without neutralizing antibodies. 100 μl of each dilution was added to each well (200 μl final volume). Plates were then incubated at 37° C., 5% CO₂ for 18-24 h.
   Day 3:
1. All wells were aspirated to remove Media D (+/−IFNAR2 Ab dilutions as indicated in the data sets/graphs). Bioassay media was added to each well (100 μl/well).
2. All the wells, except control wells, were challenged with EMC virus. (100 μl EMC/well →200 μl final volume in Bioassay Medium)
   WISH cells:
      5 μl in 1 ml=1 MOI
      For each plate requiring virus (100 μl/well=7 ml)
         35 μl (undiluted virus) in 6965 μl bioassay media
3. Cells are again incubated at 37° C., 5% CO₂ for 18-24 hours (in an incubator dedicated for viral work).
   Day 4:
1. Media was aspirated and stained with 0.5% crystal violet (290 μl per well) for 10-30 min and then rinsed with distilled water (2×).
2. Plates were read at 540 nm after drying.

### RESULTS

Neutralization of Interferons by IFNAR2 Antibodies in Anti-Viral Bioassays

Antibodies generated against IFNAR2 were tested for their ability to neutralize the anti-viral effect of 1000 U/ml of IFN-α with respect to virus-challenged WISH fibroblast cells. Data for antibodies 1922 and 1923 are shown in Table A and graphically depicted in FIG. 1. Control experiments consisted of (i) cells grown in the presence of IFN-α; (ii) cells grown in the presence of IFN-α and a known blocking anti-IFN-α antibody; (iii) growth of unstimulated cells (i.e., no addition of Type I interferons); (iv) growth of cells in the absence of virus. A third antibody, antibody 1924, was also tested for its ability to neutralize interferon-α. Antibody 1924 was capable of neutralizing interferon-α, albeit with a less robust activity than antibody 1922 and 1923 when tested at the same antibody concentration (data not shown). Antibody 1924 was also generically characterized in Chuntharapai et al., J. Immunol. (1999), 163:766-773 (antibody referred to as “3B2” in Table 1.) Hybridoma cell line expressing antibody 1924 has now been deposited at the ATCC, as indicated below.

TABLE A

| | | replicate | replicate | replicate | |
|---|---|---|---|---|---|
| | ug/ml | 1 | 2 | 3 | average |
| Antibody 1922 | 40 | 0.276 | 0.203 | 0.265 | 0.248 |
| (1000 U/ml IFN-α) | 20 | 0.146 | 0.11 | 0.079 | 0.111666667 |
| | 10 | 0.502 | 0.243 | 0.342 | 0.362333333 |
| | 50 | 0.578 | 0.254 | 0.106 | 0.312666667 |
| | 2.5 | 0.745 | 0.75 | 0.707 | 0.734 |
| | 1.25 | 1.084 | 1.396 | 1.107 | 1.195666667 |
| | 0.63 | 1.495 | 1.265 | 1.243 | 1.334333333 |
| | 0.31 | 1.738 | 1.815 | 1.8662 | 1.8064 |
| Antibody 1923 | 40 | 0.802 | 0.366 | 0.367 | 0.511666667 |
| (1000 U/ml IFN-α) | 20 | 0.134 | 0.179 | 0.563 | 0.292 |
| | 10 | 0.486 | 0.679 | 0.759 | 0.641333333 |
| | 50 | | 0.951 | 1.253 | 1.102 |
| | 2.5 | 1.541 | 0.943 | 1.435 | 1.306333333 |
| | 1.25 | 1.086 | 1.546 | 1.631 | 1.421 |
| | 0.63 | 1.336 | 1.684 | 1.572 | 1.530666667 |
| | 0.31 | 2.017 | 2.39 | 1.72 | 2.042333333 |
| controls | IFN-α | 1.936 | 2.083 | 2.177 | 2.065333333 |
| | IFN-α + anti- | 0.206 | 0.628 | | 0.417 |
| | IFN-α ab | | | | |
| | no stimulation | 0.088 | 0.143 | 0.139 | 0.123333333 |
| | no virus | 1.983 | 1.845 | 2.107 | 1.978333333 |

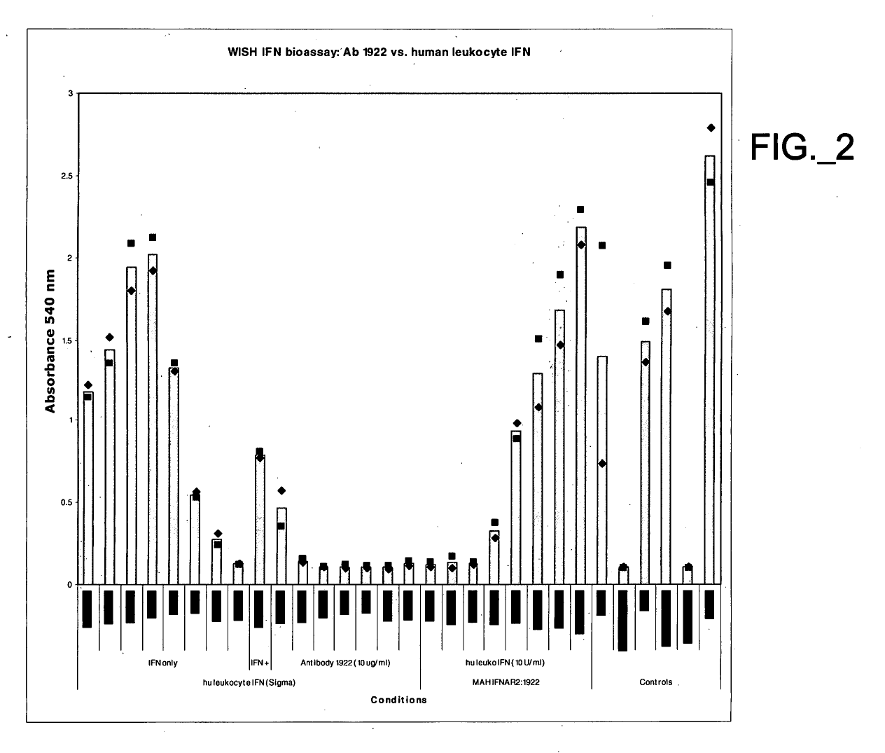

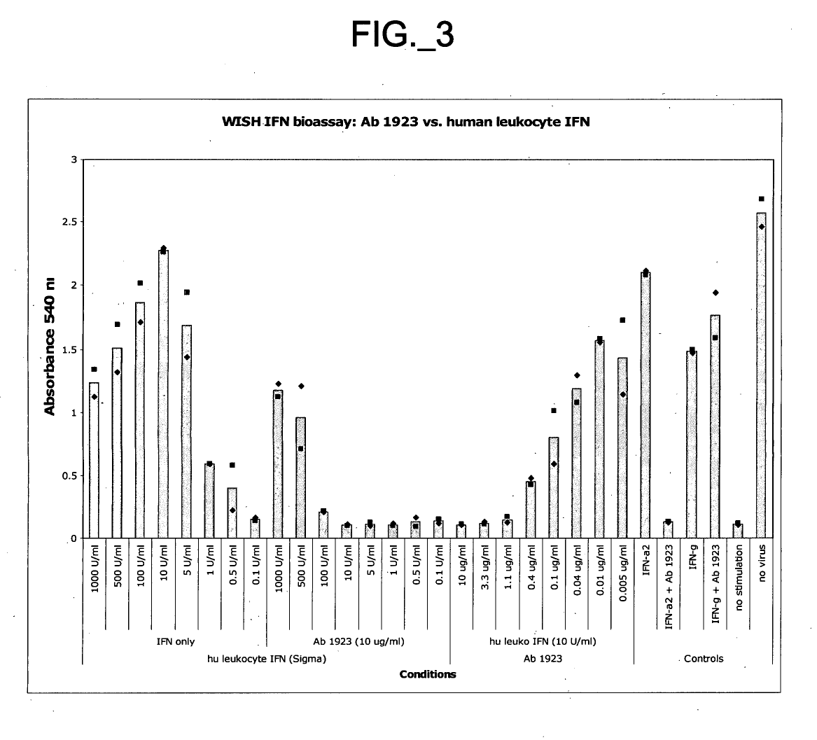

Antibodies generated against IFNAR2 were also tested for their ability to neutralize the anti-viral effect of human leukocyte interferon at various concentrations with respect to virus-challenged WISH fibroblast cells. Data for antibodies 1922 are shown in Table B and graphically depicted in FIG. 2. Data for antibodies 1923 are shown in Table C and graphically depicted in FIG. 3. Control experiments consisted of (i) cells grown in the presence of interferon α2 (1000 U/ml; Sigma); (ii) cells grown in the presence of interferon α2 (1000 U/ml; Sigma) and Ab 1922 or Ab 1923 (10 ug/ml); (iii) cells grown in the presence of IFN-γ(10 U/ml; PBL); (iv) cells grown in the presence of IFN-γ(10 U/ml) and Antibody 1922 or 1923 (10 ug/ml); (v) growth of unstimulated cells (i.e., no addition of Type I interferons); (vi) growth of cells in the absence of virus.

TABLE B

| | | | | replicate | replicate | |
|---|---|---|---|---|---|---|
| | | | | 1 | 2 | average |
| hu leukocyte | IFN only | 1000 | U/ml | 1.219 | 1.145 | 1.182 |
| IFN | | 500 | U/ml | 1.515 | 1.358 | 1.4365 |
| | | 100 | U/ml | 1.803 | 2.083 | 1.943 |
| | | 10 | U/ml | 1.918 | 2.118 | 2.018 |
| | | 5 | U/ml | 1.303 | 1.356 | 1.3295 |
| | | 1 | U/ml | 0.563 | 0.531 | 0.547 |
| | | 0.5 | U/ml | 0.314 | 0.238 | 0.276 |
| | | 0.1 | U/ml | 0.127 | 0.122 | 0.1245 |
| | IFN + Ab 1922 | 1000 | U/ml | 0.768 | 0.804 | 0.786 |
| | (10 ug/ml) | 500 | U/ml | 0.575 | 0.352 | 0.4635 |
| | | 100 | U/ml | 0.137 | 0.152 | 0.1445 |
| | | 10 | U/ml | 0.103 | 0.107 | 0.105 |
| | | 5 | U/ml | 0.096 | 0.121 | 0.1085 |
| | | 1 | U/ml | 0.098 | 0.115 | 0.1065 |
| | | 0.5 | U/ml | 0.095 | 0.113 | 0.104 |
| | | 0.1 | U/ml | 0.112 | 0.14 | 0.126 |
| Ab 1922 | hu leuko IFN | 10 | ug/ml | 0.105 | 0.136 | 0.1205 |
| | (10 U/ml) | 3.3 | ug/ml | 0.097 | 0.167 | 0.132 |
| | | 1.1 | ug/ml | 0.119 | 0.137 | 0.128 |
| | | 0.4 | ug/ml | 0.279 | 0.372 | 0.3255 |
| | | 0.1 | ug/ml | 0.982 | 0.879 | 0.9305 |
| | | 0.04 | ug/ml | 1.083 | 1.507 | 1.295 |
| | | 0.01 | ug/ml | 1.47 | 1.895 | 1.6825 |
| | | 0.005 | ug/ml | 2.074 | 2.284 | 2.179 |

| | | | | |
|---|---|---|---|---|
| Controls | IFN-α2 (1000 U/ml) | 0.731 | 2.07 | 1.4005 |
| | IFN-α2 (1000 U/ml) + | 0.106 | 0.1 | 0.103 |
| | Ab 1922 | | | |
| | IFN-γ (10 U/ml) | 1.361 | 1.612 | 1.4865 |
| | IFN-γ (10 U/ml) + | 1.673 | 1.946 | 1.8095 |
| | Ab 1922 | | | |
| | no stimulation | 0.109 | 0.096 | 0.1025 |
| | no virus | 2.787 | 2.457 | 2.622 |

TABLE C

| | | | | replicate | replicate | |
|---|---|---|---|---|---|---|
| | | | | 1 | 2 | average |
| hu leukocyte | IFN only | 1000 | U/ml | 1.122 | 1.342 | 1.232 |
| IFN (Sigma) | | 500 | U/ml | 1.321 | 1.693 | 1.507 |
| | | 100 | U/ml | 1.714 | 2.016 | 1.865 |
| | | 10 | U/ml | 2.291 | 2.257 | 2.274 |
| | | 5 | U/ml | 1.44 | 1.94 | 1.69 |
| | | 1 | U/ml | 0.589 | 0.593 | 0.591 |
| | | 0.5 | U/ml | 0.221 | 0.578 | 0.3995 |
| | | 0.1 | U/ml | 0.165 | 0.137 | 0.151 |
| | IFN + Ab 1923 | 1000 | U/ml | 1.225 | 1.122 | 1.1735 |
| | (10 ug/ml) | 500 | U/ml | 1.205 | 0.708 | 0.9565 |
| | | 100 | U/ml | 0.209 | 0.215 | 0.212 |
| | | 10 | U/ml | 0.11 | 0.101 | 0.1055 |
| | | 5 | U/ml | 0.097 | 0.128 | 0.1125 |
| | | 1 | U/ml | 0.115 | 0.1 | 0.1075 |
| | | 0.5 | U/ml | 0.165 | 0.092 | 0.1285 |
| | | 0.1 | U/ml | 0.121 | 0.154 | 0.1375 |
| Ab 1923 | hu leuko IFN | 10 | ug/ml | 0.105 | 0.109 | 0.107 |
| | (10 U/ml) | 3.3 | ug/ml | 0.129 | 0.11 | 0.1195 |
| | | 1.1 | ug/ml | 0.123 | 0.171 | 0.147 |
| | | 0.4 | ug/ml | 0.479 | 0.424 | 0.4515 |
| | | 0.1 | ug/ml | 0.591 | 1.009 | 0.8 |
| | | 0.04 | ug/ml | 1.296 | 1.074 | 1.185 |
| | | 0.01 | ug/ml | 1.557 | 1.582 | 1.5695 |
| | | 0.005 | ug/ml | 1.139 | 1.729 | 1.434 |

| | | | | |
|---|---|---|---|---|
| Controls | IFN-α2 (1000 U/ml) | 2.116 | 2.081 | 2.0985 |
| | IFN-α2 (1000 U/ml) + | 0.127 | 0.131 | 0.129 |
| | Ab 1923 | | | |
| | IFN-γ (10 U/ml) | 1.473 | 1.494 | 1.4835 |
| | IFN-γ (10 U/ml) + | 1.945 | 1.586 | 1.7655 |
| | Ab 1923 | | | |
| | no stimulation | 0.104 | 0.12 | 0.112 |
| | no virus | 2.459 | 2.688 | 2.5735 |

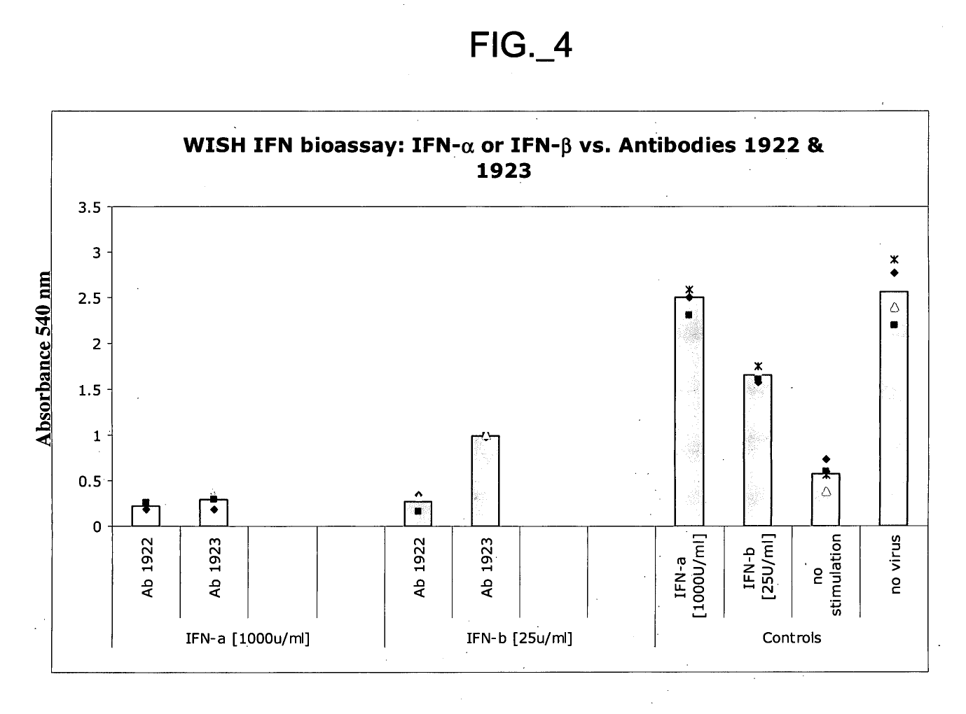

Antibodies were also tested for ability to neutralize the anti-viral protective effects of IFN-β. Data for antibodies 1922 and 1923, tested at a concentration of 10 ug/ml against either IFN-α(at 1000 U/ml) or IFN-β(at 25 U/ml), are depicted in Table D below and FIG. 4. Control experiments also consisted of: (i) cell viability in the presence of IFN-60 only; (ii) cell growth in the presence of IFN-β only; (iii) viability of unstimulated cells; (iv) viability of cells in the absence of virus.

TABLE D

| | | replicate | replicate | replicate | replicate | |
|---|---|---|---|---|---|---|
| | | 1 | 2 | 3 | 4 | Ave |
| IFN-α | Ab 1922 | 0.186 | 0.258 | | | 0.222 |
| [1000 u/ml] | Ab 1923 | 0.177 | 0.297 | 0.383 | | 0.285666667 |
| IFN-β | Ab 1922 | 0.337 | 0.163 | 0.317 | | 0.272333333 |
| [25 u/ml] | Ab 1923 | 0.974 | 0.99 | 1 | | 0.988 |
| Controls | IFN-α | 2.505 | 2.313 | 2.587 | 2.593 | 2.4995 |
| | [1000 U/ml] | | | | | |
| | IFN-β [25 U/ml] | 1.566 | 1.611 | 1.691 | 1.746 | 1.6535 |
| | no stimulation | 0.73 | 0.591 | 0.379 | 0.563 | 0.56575 |
| | no virus | 2.768 | 2.201 | 2.392 | 2.911 | 2.568 |

Antibody 1922 was also tested for ability to neutralize the anti-viral protective effects of IFN-β (PBL, catalog no. 1400-2) over a range of interferon concentrations. Data are depicted in Table E below and FIG. 5. Control experiments also consisted of: (i) cell viability in the presence of IFN-α only (@ 1000 U/ml); (ii) cell viability in IFN-α(@ 1000 U/ml) and antibody 1922 (@ 10 ug/ml); (iii) viability of unstimulated cells; and (iv) viability of cells in the absence of virus.

TABLE E

| | | | replicate | replicate | replicate | |
|---|---|---|---|---|---|---|
| | Conditions | | 1 | 2 | 3 | average |
| Ab 1922 (10 ug/ml) + | 500 | U/ml | 0.333 | 0.123 | 0.184 | 0.213333333 |
| IFN-β (PBL) | 250 | U/ml | 0.192 | 0.388 | 0.251 | 0.277 |
| | 100 | U/ml | 0.268 | 0.125 | 0.155 | 0.182666667 |
| | 50 | U/ml | 0.234 | 0.329 | 0.122 | 0.228333333 |
| | 10 | U/ml | 0.177 | 0.196 | 0.103 | 0.158666667 |
| | 1 | U/ml | 0.084 | 0.103 | 0.116 | 0.101 |

| | | | | | |
|---|---|---|---|---|---|
| Controls | IFN-α (1000 U/ml) | 2.127 | 1.98 | 1.79 | 1.965666667 |
| | IFN-α + Ab 1922 | 0.12 | 0.126 | 0.207 | 0.151 |
| | no stimulation | 0.185 | 0.281 | 0.172 | 0.212666667 |
| | no virus | 2.47 | 2.617 | 2.738 | 2.608333333 |

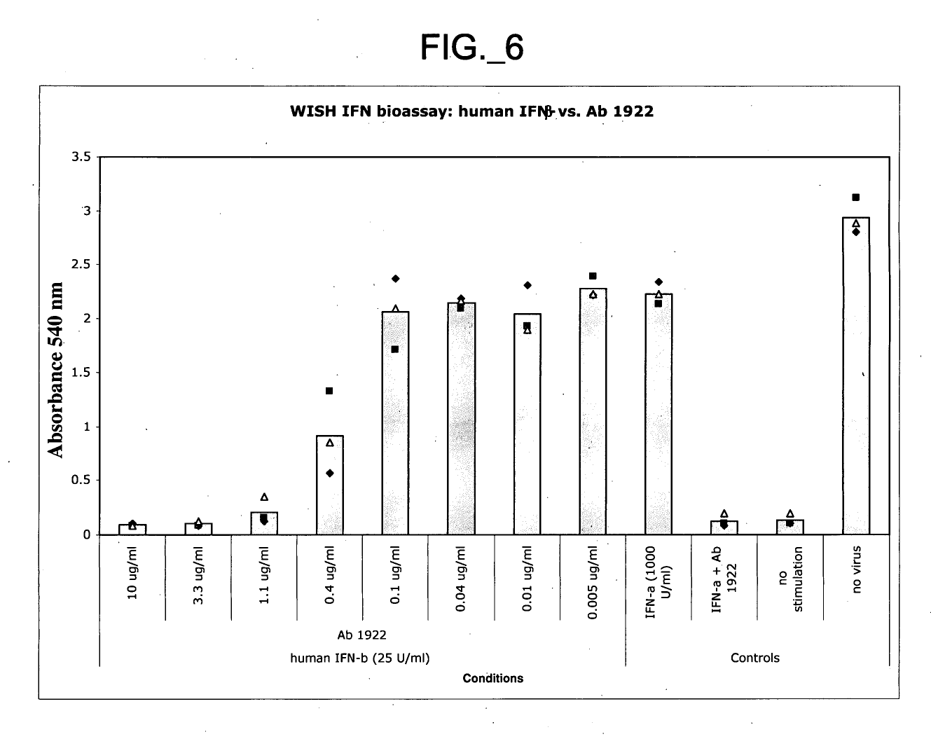

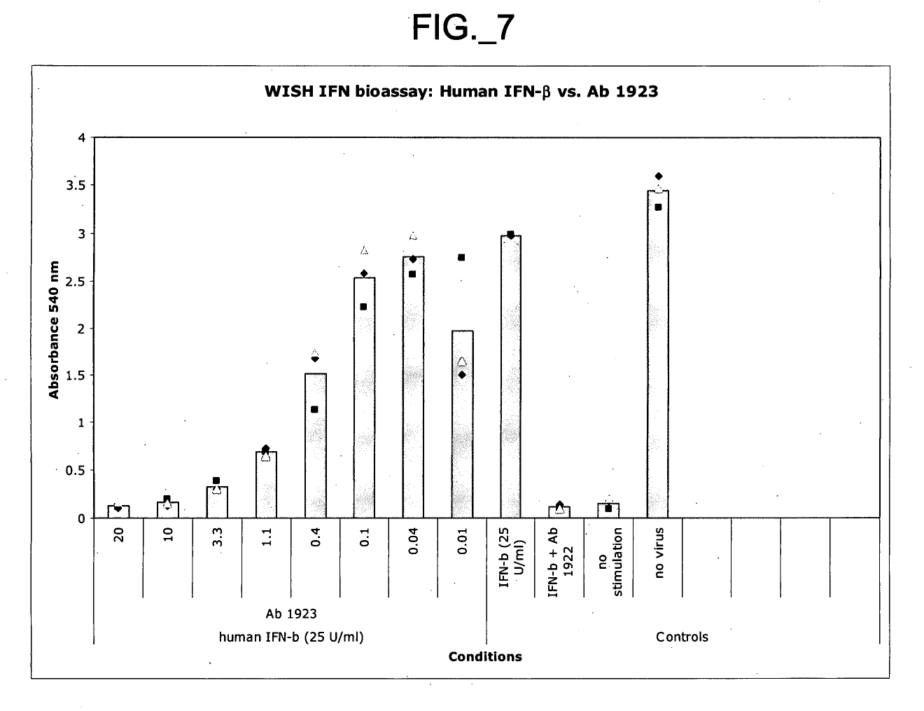

Antibody 1922 and 1923 were also tested for ability to neutralize the anti-viral protective effects of IFN-β (PBL, catalog no. 11400-2) over a range of antibody concentrations. Data for antibody 1922 are depicted in Table F below and FIG. 6. Data for antibody 1923 are depicted in Table G below and FIG. 7. Control experiments also consisted of: (i) cell growth in the presence of interferon-α(1000 U/ml); (ii) cell viability in the presence of interferon-α and Ab 1922 (Table F), or interferon-β and Ab 1922 (Table G); (iii) cell viability of unstimulated cells; and (iv) viability of cells in the absence of virus.

TABLE F

| | | | | replicate | replicate | replicate | |
|---|---|---|---|---|---|---|---|
| | | | | 1 | 2 | 3 | average |
| human IFN-β | Ab | 10 | ug/ml | 0.105 | 0.079 | 0.082 | 0.088666667 |
| (25 U/ml) | 1922 | 3.3 | ug/ml | 0.086 | 0.087 | 0.122 | 0.098333333 |
| | | 1.1 | ug/ml | 0.123 | 0.158 | 0.348 | 0.209666667 |
| | | 0.4 | ug/ml | 0.568 | 1.329 | 0.855 | 0.917333333 |
| | | 0.1 | ug/ml | 2.369 | 1.711 | 2.093 | 2.057666667 |
| | | 0.04 | ug/ml | 2.187 | 2.087 | 2.162 | 2.145333333 |
| | | 0.01 | ug/ml | 2.304 | 1.93 | 1.89 | 2.041333333 |
| | | 0.005 | ug/ml | 2.21 | 2.389 | 2.227 | 2.275333333 |

| | | | | | |
|---|---|---|---|---|---|
| Controls | IFN-α (1000 U/ml) | 2.334 | 2.127 | 2.223 | 2.228 |
| | IFN-α + Ab 1922 | 0.084 | 0.106 | 0.194 | 0.128 |
| | no stimulation | 0.098 | 0.103 | 0.198 | 0.133 |
| | no virus | 2.804 | 3.118 | 2.886 | 2.936 |

TABLE G

| | | | replicate | replicate | replicate | |
|---|---|---|---|---|---|---|
| | | ug/ml | 1 | 2 | 3 | average |
| human IFN-β | Ab | 20 | 0.11 | 0.117 | 0.168 | 0.131666667 |
| (25 U/ml) | 1923 | 10 | 0.131 | 0.2 | 0.149 | 0.16 |
| | | 3.3 | 0.307 | 0.389 | 0.301 | 0.332333333 |
| | | 1.1 | 0.72 | 0.685 | 0.646 | 0.683666667 |
| | | 0.4 | 1.678 | 1.137 | 1.741 | 1.518666667 |
| | | 0.1 | 2.578 | 2.211 | 2.82 | 2.536333333 |
| | | 0.04 | 2.729 | 2.56 | 2.969 | 2.752666667 |
| | | 0.01 | 1.506 | 2.743 | 1.649 | 1.966 |
| Controls | | IFN-β (25 U/ml) | 2.97 | 2.982 | | 2.976 |
| | | IFN-β + Ab 1922 | 0.137 | 0.11 | 0.097 | 0.114666667 |
| | | no stimulation | 0.176 | 0.096 | 0.193 | 0.155 |
| | | no virus | 3.597 | 3.269 | 3.463 | 3.443 |

Binding Affinity of Antibodies to IFNAR2 Assessed by Biacore Analysis

Binding affinities of Ab 1922 and 1923 to human IFNAR2.ECD.IgG1 were determined by Biacore. While no binding was observed to murine IgG1 and IgG2a, Ab 1922 and 1923 showed high binding affinity to IFNAR2.

TABLE H

| ANTIBODY | BINDING AFFINITY |
|---|---|
| Control antibody 1 | no binding |
| Control antibody 2 | no binding |
| Ab 1922 | 58 pM |
| Ab 1923 | 280 pM |
| Control antibody 3 | no binding |

The following hybridomas have been deposited with the American Type Culture Collection, 12301 Parklawn Drive, Rockville, Md., USA (ATCC):

| Cell Lines | ATCC Accession No. | Deposit Date |
|---|---|---|
| Antibody 1922 (1C7.8.8) | PTA-6242 | Oct. 5, 2004 |
| Antibody 1923 (2B4.10.6) | PTA-6243 | Oct. 5, 2004 |
| Antibody 1924 (3B2.5.7) | PTA-6244 | Oct. 5, 2004 |

These deposits were made under the provisions of the Budapest Treaty on the International Recognition of the Deposit of Microorganisms for the Purpose of Patent Procedure and the Regulations thereunder (Budapest Treaty). This assures maintenance of a viable deposit for 30 years from the date of deposit. These cell lines will be made available by ATCC under the terms of the Budapest Treaty, and subject to an agreement between Genentech, Inc. and ATCC, which assures permanent and unrestricted availability of the cell lines to the public upon issuance of the pertinent U.S. patent or upon laying open to the public of any U.S. or foreign patent application, whichever comes first, and assures availability of the cell lines to one determined by the U.S. Commissioner of Patents and Trademarks to be entitled thereto according to 35 USC § 122 and the Commissioner's rules pursuant thereto (including 37 CFR § 1.14 with particular reference to 886OG 638).

The assignee of the present application has agreed that if the deposited cell lines should be lost or destroyed when cultivated under suitable conditions, they will be promptly replaced on notification with a specimen of the same cell line. Availability of the deposited cell lines is not to be construed as a license to practice the invention in contravention of the rights granted under the authority of any government in accordance with its patent laws.

1. An isolated antibody comprising at least one hypervariable (HVR) sequence selected from the group consisting of HC-HVR1, HC-HVR2, HC-HVR3, LC-HVR1, LC-HVR2 and LC-HVR3 of an antibody produced by hybridoma cell line deposited at American Type Culture Collection (ATCC) under Accession No. PTA-6242, PTA-6243 or PTA-6244, wherein said isolated antibody binds human interferon alpha receptor 2 (IFNAR2).

2. An isolated antibody comprising heavy and/or light chain variable domain sequence of an antibody produced by hybridoma cell line deposited at American Type Culture Collection (ATCC) under Accession No. PTA-6242, PTA-6243 or PTA-6244, wherein said isolated antibody binds human interferon alpha receptor 2 (IFNAR2).

3. An immunoglobulin polypeptide comprising at least one hypervariable (HVR) sequence selected from the group consisting of HC-HVR 1, HC-HVR2, HC-HVR3, LC-HVR 1, LC-HVR2 and LC-HVR3 of an antibody produced by hybridoma cell line deposited at American Type Culture Collection (ATCC) under Accession No. PTA-6242, PTA-6243 or PTA-6244, wherein said immunoglobulin polypeptide binds human interferon alpha receptor 2 (IFNAR2).

4. An immunoglobulin polypeptide comprising heavy and/or light chain variable domain sequence of an antibody produced by hybridoma cell line deposited at American Type Culture Collection (ATCC) under Accession No. PTA-6242, PTA-6243 or PTA-6244, wherein said immunoglobulin polypeptide binds human interferon alpha receptor 2 (IFNAR2).

5. An isolated antibody that binds to the same epitope on human IFNAR2 as the antibody produced by hybridoma cell line deposited at American Type Culture Collection (ATCC) under Accession No. PTA-6242, PTA-6243 or PTA-6244.

6. An isolated antibody that competes with the antibody produced by hybridoma cell line deposited at American Type Culture Collection (ATCC) under Accession No. PTA-6242, PTA-6243 or PTA-6244 for binding to human IFNAR2.

7. The antibody of claim 1, wherein the antibody inhibits anti-viral activity of human leukocyte interferon.

8. The antibody of claim 1, wherein the antibody inhibits anti-viral activity of human interferon alpha.

9. The antibody of claim 1, wherein at least about 10 ug/ml of the antibody in full length IgG form inhibits at least about 25% of anti-viral activity of from about 0.5 U/ml to about 1000 U/ml of human leukocyte interferon.

10. The antibody of claim 9, wherein the leukocyte interferon is about 10 U/ml.

11. The antibody of claim 1, wherein at least about 10 ug/ml of the antibody in full length IgG form inhibits at least about 25% of anti-viral activity of about 1000 U/ml of interferon α.

12. The antibody of claim 1, wherein at least about 0.01 ug/ml of the antibody in full length IgG form inhibits at least about 25% of anti-viral activity of about 25 U/ml of interferon β.

13. The antibody of claim 12, wherein the antibody concentration is at least about 10 ug/ml.

14. The antibody of claim 1, wherein at least about 10 ug/ml of the antibody in full length IgG form inhibits at least about 25% of anti-viral activity of about 25 U/ml of interferon β.

15. The antibody of claim 1, wherein the full length IgG form of the antibody specifically binds human IFNAR2 with a binding affinity of 300 pM or better.

16. The antibody of claim 15 wherein the binding affinity is 280 pM or better.

17. The antibody of claim 16 wherein the binding affinity is 200 pM or better.

18. The antibody of claim 17 wherein the binding affinity is 100 pM or better.

19. The antibody of claim 18 wherein the binding affinity is 60 pM or better.

20. The antibody of claim 1, wherein the antibody blocks anti-viral activity of interferon α and interferon β at substantially equivalent antibody titer.

21. The antibody of claim 1, wherein an equivalent amount of the antibody is capable of blocking at least 75% of anti-viral activity of a first Type I interferon and a second Type I interferon, wherein the interferons are each administered at their respective optimal anti-viral amount in a WISH cell bioassay, and wherein the second Type I interferon is interferon β.

22. The IFNAR2 antibody of claim 21 wherein the first Type I interferon is an interferon α.

23. The IFNAR2 antibody of claim 21 wherein the first Type I interferon is human leukocyte interferon.

24. The isolated antibody of claim 1, wherein the antibody is not an antibody produced by hybridoma cell line having ATCC Deposit No. HB-12426, 12427 and/or 12428, or an IFNAR2 antibody described on pages 10895 to 10899 in Journal of Biological Chemistry, Volume 268 published in 1993, or an isolated IFNAR2 antibody disclosed in PCT Publications WO96/33735, WO96/34096, WO9741229, European Patent Nos. 588177 B1, 927252, 676413, and/or U.S. Pat. Nos. 6,458,932 and 6,136,309.

25. The isolated antibody of claim 1, wherein the antibody does not compete for binding to human IFNAR2 with an antibody produced by hybridoma cell line having ATCC Deposit No. HB-12426, 12427 and/or 12428, or an IFNAR2 antibody described on pages 10895 to 10899 in Journal of Biological Chemistry, Volume 268 published in 1993, or an isolated IFNAR2 antibody disclosed in PCT Publications WO96/33735, WO96/34096, WO9741229, European Patent Nos. 588177 B1, 927252, 676413, and/or U.S. Pat. Nos. 6,458,932 and 6,136,309.

26. The isolated antibody of claim 1, wherein the antibody does not bind to the same epitope on IFNAR2 as an antibody produced by hybridoma cell line having ATCC Deposit No. HB-12426, 12427 and/or 12428, or an IFNAR2 antibody described on pages 10895 to 10899 in Journal of Biological Chemistry, Volume 268 published in 1993, or an isolated IFNAR2 antibody disclosed in PCT Publications WO96/33735, WO96/34096, WO9741229, European Patent Nos. 588177 B1, 927252, 676413, and/or U.S. Pat. Nos. 6,458,932 and 6,136,309.

27. An IFNAR2 antibody encoded by an antibody coding sequence of hybridoma cell line deposited at the American Type Culture Collection (ATCC) under Accession No. PTA-6242, PTA-6243 or PTA-6244.

28. A nucleic acid molecule encoding the antibody of claim 1.

29. A host cell comprising a nucleic acid sequence encoding the antibody of claim 1.

30. A cell line capable of producing the IFNAR2 antibody of claim 1.

31. A method of producing the antibody of claim 1, comprising culturing a host cell comprising a nucleic acid encoding the antibody under conditions wherein the antibody is produced.

32. A composition comprising an effective amount of the antibody of claim 1, and a carrier.

33. A method of diagnosing presence of IFNAR2 in a sample, comprising contacting the sample with an antibody of claim 1.

34. A method for treatment of a disease or condition associated with expression of IFN-α, β and/or IFNAR2, the method comprising administering to the patient an effective amount of an antibody of claim 1.

35. The method of claim 34, wherein the patient is a mammalian patient.

36. The method of claim 34, wherein the patient is human.

37. The method of claim 34, wherein the disease is an autoimmune disease.

38. The method of claim 37, wherein the disease is selected from the group consisting of insulin-dependent diabetes mellitus (IDDM); systemic lupus erythematosus (SLE), autoimmune thyroiditis, Sjogren's syndrome, psoriasis, inflammatory bowel disease (e.g., ulcerative colitis, Crohn's disease), rheumatoid arthritis and IgA nephropathy.
